(19) United States
(12) Patent Application Publication
(10) Pub. No.: US 2026/0229320 A1
(43) Pub. Date: Aug. 6, 2026

(54) ENHANCED HUMAN-COMPUTER INTERACTION SYSTEMS

(71) Applicant: ASEDASCIENCES AG, Schindellegi (CH)

(72) Inventors: Bradley CALVIN, North Potomac, MD (US); Bartlomiej RAJWA, West Lafayette, IN (US)

(73) Assignee: ASEDASCIENCES AG, Schindellegi (CH)

(21) Appl. No.: 19/150,164

(22) PCT Filed: Jan. 23, 2024
(86) PCT No.: PCT/US2024/012520

§ 371 Date: Jul. 22, 2025

Related U.S. Application Data
(60) Provisional application No. 63/440,548, filed on Jan. 23, 2023.

(51) Int. Cl. G16C 20/80; G06F 16/26; G06F 16/28; G16C 20/90

(52) U.S. Cl. CPC G16C 20/80; G06F 16/26; G06F 16/287; G16C 20/90

(57) ABSTRACT

Improved human-computer interaction methods, systems and displays are provided for integrating, displaying, manipulating and studying complex multiparameter information about chemical and biochemical compounds, including physical, chemical and biological properties. Systems and displays are provided in which compounds are visualized as points in a multiparametric display space according to their similarity and/or differences from other compounds. Methods for calculating distance metrics based on diverse properties are provided. Further provided are methods for displaying information on individually selected compounds in a universe of compounds in the display space. VR implementations are provided that enhance visualization of relations between compounds. The methods, systems and displays are useful for many purposes including drug discovery and development.

### FIELD OF THE INVENTION

The invention herein described in part relates to the fields of human-computer interaction and interactive displays for investigating and understanding diverse chemical and biological information datasets, and the relationships between physical and chemical properties of compounds and biological effects.

### GOVERNMENT FUNDING

No government funds were used in making the invention herein disclosed and claimed.

### INTRODUCTION

Over the past few decades, artificial intelligence and machine learning (AI/ML) have gained widespread acceptance in numerous fields, including biomedical research. Human-Computer Interaction (HCI) is a specialized area of research and application that merges AI/ML, the use of big data, and knowledge of how humans and computers interact to create interfaces that allow for more effective interaction between humans and computer-processed data HCI is an ever-evolving field of study that explores how to design, develop, implement, and evaluate interactive computer systems that are centered on the needs of humans. Its goal is to maximize the usability, effectiveness, and efficiency of the interfaces between users, computers, and data. Within the realm of HCI, “interaction” refers to the ways in which humans communicate and interact with computers to complete tasks, while “interface” refers to the technical means, platforms, or applications that enable a specific model of interaction (Gurcan et al., 2021).

HCI can play a crucial role in facilitating the visualization, communication, and decision-making processes related to the analysis of complex data sets. For instance, the process of data visualization enables researchers to gain a more comprehensive understanding of the underlying patterns and relationships within data, thereby allowing for more informed decision-making, such as regarding the selection, prioritization, and classification of chemical compounds based on their effect on various diverse, multiparameter biochemical and biological processes within live cells, organs or whole organisms. By employing advanced HCI techniques and tools, researchers can interact with data in real time, manipulate it, and view it from multiple perspectives, all of which can help uncover hidden insights and trends that may not be immediately apparent through other means. Ultimately, the goal of incorporating HCI into the data analysis process is to improve the overall efficiency and accuracy of scientific research by empowering researchers to make better decisions and gain deeper insights into the complex systems they am studying.

In the field of drug discovery and related areas such as organic chemistry, medicinal chemistry, and toxicology, an effective HCI system paired with an appropriate biological data-collecting strategy can serve as a powerful tool for suggesting, enabling, and empowering users to make informed decisions about which compounds to select, prioritize, modify or discard, and how these compounds should be ranked in terms of the associated risk.

By utilizing an HCI system, users can visualize relationships, similarities, properties, and interactions among various biologically active compounds, which can aid in selecting and ranking and selecting those compounds for further modification or investigation. Additionally, an effective HCI system that can incorporate and visualize multiple different and complex biological data-collecting strategies involving multi-omics data, that is, data originating from phenomics, cytomics, proteomics, lipidomics, transcriptomics, and other methodologies, can further enhance and accelerate the effectiveness of the decision making process by enabling users to make more informed and better decisions regarding the selection and prioritization for further exploration and development of compounds from based on complex data sets.

Therefore, the combination of an appropriately designed HCI system with an efficient data collection and integration technique for visualizing the cause-effect relationship between drugs and complex biological processes within cells, collections of cells, organs or whole organisms can considerably improve the drug discovery process by enabling scientists to make better-informed judgments regarding the selection and ranking of compounds for further study.

One of the most crucial facets of HCI is displaying scientific data or, more generally, visual analytics (VA). In order to enable visualization, these scientific data sets typically require automatic data processing steps, including dimensionality reduction, clustering, and classification. Typically, scientific visualization employs 2D or 3D representations of data clouds, where each point can be interpreted as a scalar or vector. Scientists utilize data visualization to identify patterns, characteristics, correlations, and anomalies (Widjojo et al., 2017).

Visualization and interpretation of chemical compound knowledge are vital for developing new medications, drug repurposing, and toxicological studies on existing and new compounds or chemicals. Chemical space is an abstract idea of describing a compound of interest in a multidimensional cartesian space in which its location is defined by a quantitative representation of its features (e.g., solubility, molecular weight, polarity, etc.). The study on the representation of compounds in multidimensional feature space results from medicinal chemists', biologists' and biochemists' longstanding interest in establishing links between the structure and function of the examined compound, particularly those with therapeutic potential (Reymond, 2015). Therefore, chemical space is a fundamental concept in chemoinformatics. It provides a framework for the study of the chemical compounds that populate, or could populate, the “chemical universe,” i.e., all possible molecules (Medina-Franco et al., 2022). The chemical space mapping tools provide an intuitive depiction of the investigated compounds, can aid in the detection of subgroups (clusters) within the data, and can be used to evaluate the data in search of potential structure-activity relationship (SAR) information (Awale et al., 2013; Gütlein et al., 2012).

The similarity between compounds must be defined for the 3D mapping process. Using a Tanimoto coefficient, which refers to the amount of chemical traits they share in common divided by the union of all features, is the most straightforward method (a percent similarity with values from 0 to 1) (Lu and Carlson, 2016).

The employed chemical data vectors can be quite complex. The webDrugCS was a web application that was formerly available for free at www.gdb.unibe.ch. It provided information about the compounds' properties via interactive color-coded 3D visualization. The webDrugCS system used DrugBank (http://www.drugbank.ca), a public database listing over 6000 chemicals now in medical usage, as FDA-approved and marketed medications or investigational drugs, as a source of information. The webDrugCS generated the display) using the user's web browser. The compound database was represented as color-coded 3D objects with locations derived from the principal component analysis (PCA) of distinct chemical fingerprints. These fingerprints describe the molecular structure and topology (42D molecular quantum numbers. MQN), structural features (34D SMILES fingerprint SMIfp), molecular shape (20D atom pair fingerprint APfp), pharmacophores (55D atom category extended atom pair fingerprint), and substructures (55D atom category extended atom pair fingerprint) (1024D binary substructure fingerprint). The visualization was generated using three.js, an open-source JavaScript library/API for animated 3D computer graphics in a web browser (http://three.org) (Awale and Reymond, 2016).

A similar system (ChemMaps), but focusing on full interactivity, has been available from the National Toxicology Program of the USDHHS (Borrel et al., 2018) The web-server navigation is driven by a chemical characteristics fingerprint that can be fully customized by a user. ChemMaps.com was built using HTML/JavaScript and the Three.js framework, which permits interactive, mouse-based, user-friendly navigation in any web browser on mobile or desktop platforms. Since all information and coordinates of the molecules have been pre-calculated, the data exploration is instantaneous. ChemMaps.com was designed to operate with the most common Web browsers (such as Firefox, Chrome. and Safari) and requires the WebGL JavaScript API.

The webDrugCS authors moved the demonstrated chemical visualization to a virtual reality engine (Probst and Reymond, 2018). As before, the utilized data was a subset of DrugBank containing only drugs designated as Approved. Experimental, or Investigational. To acquire the 3D coordinates for the visual depiction of molecules in the VR space, their respective MQN fingerprints with 42 dimensions were determined. Similarly to the previously demonstrated system, the PCA is then used to embed the fingerprint vectors within a 3D space. The colorization of the data points extends the visualization into the fourth dimension. The visualization was developed using the Unity game engine, which is capable of visualizing enormous volumes of scientific data or point clouds in a virtual reality context. In addition, the Virtual Reality Toolkit (VRTK) for Unity was used to support development unrelated to a particular virtual reality headset (Probst and Reymond, 2018). Consequently, the prototype was compatible with both the Oculus Rift and HTC Vive (through SteamVR) headsets. Additionally, Google Daydream and Ximmerse View were partially supported.

The straightforward PCA-based 3D embedding can be replaced by several non-linear approaches based on manifold learning. Also, the embedding results can be represented as a point cloud, a network, or a minimal spanning tree for clarity of the display (Probst and Reymond, 2020). Non-linear principal component analysis (NLPCA), t-distributed stochastic neighbor embedding (t-SNE), uniform manifold approximation and projection (UMAP), probabilistic generative topographic maps (GTM), self-organizing maps (SOM), or graphs based on locality-sensitive hashing would be the obvious choice for the embedding methodology (LSH) (Probst and Reymond, 2020, 2018; Karlov et al., 2019). All of these technologies were shown within the context of chemical data database management and visualization. (Bawa et al., 2005; Probst and Reymond, 2020). The developed chemical space maps are an important tool for drug discovery efforts (Sebastián-Pérez et al., 2017).

Although most of the work of compound landscape visualization has been performed using purely chemical data, some effort has been reported on the integration of biochemical information as well. For instance, Donmez et al. (2020) present a program (called iBioProVis) that employs a map-based method to embed active compounds in the context of their cognate target proteins and interactively visualizes these relationships in 2D space based on the structural descriptors of the compounds. In principle, these compound-protein interactions could be shown in virtual reality 3D space; however, the authors did not demonstrate this capability. Other researchers focused on several other biochemical properties. Capecchi and Reymond (2020) analyzed the Natural Products Atlas (NPAtlas), a database of 25,523 NPs of bacterial or fungal origin, using their MAP4 fingerprint (MinHashed Atom Pair fingerprint with a diameter of four bonds).

The chemical maps are typically created for small molecules, but they have also been demonstrated for the visualization of proteins. The maps enabling nearest neighbor searches can be used to identify closely related biomolecules, from small peptides to enzymes and large multiprotein complexes such as virus particles (Jin et al., 2015).

3D data visualization has also been employed in biology (Turban and Gümüş, 2022). It is especially popular among scientists studying biological interactions, which can be represented as networks. Most of the published work has been accomplished utilizing 2D/pseudo-3D visualization engines that display the data on a computer screen, such as Cytoscape and Gephi. In addition, there are several JavaScript. Python, and R network visualization libraries (sigma.js, iGraph, etc.). Nevertheless, there are also notable examples of 3D, stereoscopic, and immersive biomolecular network visualization tools that are open source, publicly accessible, and compatible with commercial hardware/software (Liluashvili et al., 2017). Of course, the network analysis typically presupposes the similarity between the nodes or starts with the known information about the association between nodes rather than discovering them from the data. However, complex biological data (such as single-cell multi-omics readout) have also been demonstrated as an input to VR 3D visualization

The visualization systems commonly used in chemical compound analysis have traditionally focused on the structural and chemical properties of the compounds. While these systems are effective in depicting molecular structures and chemical characteristics, they are limited in their ability to represent the broader biological context in which such compounds are typically used. As a result, these traditional visualization methods do not provide information about the complex interplay between a compound's structure and its biological function, which is essential for a comprehensive understanding of how these compounds interact within biological systems and contribute to their overall function and efficacy. Furthermore, conventional visualization techniques for chemical data lack the ability to dynamically represent the high dimensionality of biological interactions, resulting in a static view of chemical compounds.

The presented invention tackles these shortcomings by introducing a visualization approach that goes beyond basic structural and chemical representation. By focusing on the biological functions and interactions of chemical compounds with living things (cells, tissues, animals), this system offers a more comprehensive and dynamic perspective. It enables a deeper comprehension of how chemical compounds operate within a biological setting, making it easier to identify potential therapeutic targets and predict side effects. The interactive nature of the platform, coupled with its capacity to incorporate a broad range of biochemical data, represents a significant leap forward from previous methods. This innovation not only enhances the precision of compound analysis but also expedites the pace of discovery in pharmaceutical research, biochemistry education, and other related fields.

### SUMMARY OF ILLUSTRATIVE EMBODIMENTS

The following enumerated paragraphs are directed to certain of the many specific embodiments of inventions herein disclosed. References in these paragraphs to the “enumerated paragraphs” refer to all of these paragraphs. The phrase “any of the foregoing or following enumerated paragraphs is inclusive and refers to all of these paragraphs A1 through F1, taken in any part or whole in any combination. The embodiments therein described are illustrative of some of the many aspects of the invention and are not intended as limiting descriptions thereof.

A full understanding of the inventions herein described can be had only by reading the entirely of the present disclosure and claims and its priority documents in light of the knowledge and insight of a person skilled in the arts to which they pertain.

Applicants reserves the right to seek patent rights on any subject matter herein disclosed and is in no way limited to the subject matter set out below.

A1. A system for displaying biological properties of compounds, comprising:
(A) One or more selectable databases of information on a plurality of chemical compounds wherein the information in the one or more selectable databases alone or in combination comprises chemical, physical and structural information and biological information for each compound;
(B) A computational device to calculate for compounds selected from one or more of the selectable databases a relative location in a biological parameter space for each of the selected compounds based on biological information for the compound in the database.
(C) A computational device to calculate for compounds selected from one or more of the selectable databases a relative location in a chemical, physical and/or structural properties parameter space for each of the selected compounds based on chemical, physical and/or structural information for the compound in the database;
(D) A computational device to display the parameter spaces of (B) and (C) and to map the selected compounds therein;
(E) One or more computational modules for human-computer interaction effective for users to:
   (a) select databases from which information is to be displayed;
   (b) select compounds to display from each selected database;
   (c) select biological, physical, chemical and/or structural parameter spaces in which compounds will be represented, including spaces based on combinations of the parameters;
   (d) select methods for calculating the location and/or distance of compounds in the parameter spaces,
   (e) adjust parameters of the parameter space, including scales.
   (f) select and display additional information about compounds;
A2. A display according to A1, where the location of compounds in the biological parameter space is determined by one or more dissimilarity and/or a similarity metrics.
A3. A display according to and one of A1 to A2, wherein the metric is the Cell Health Index.
A4. A display according to any one of A1 to A3, wherein biological assay measures are stretched between 0 and 1, where 0 denotes lack of response, and 1 denotes maximal observable response, and the similarity between responses is constructed as a distance between these bound response vectors:

$${D}_{1} = {( {\sum }_{i = 1}^{n} {( log ( \frac{1}{1 - {p}_{i}} ) + log ( {p}_{i} ) - log ( \frac{1}{1 - {q}_{i}} ) - log ( {q}_{i} ) )}^{2} )}^{\frac{1}{2}}$$

where p and q are the vectors describing the compounds.

A5. A display according to A1, wherein the similarity between two compounds that demonstrate ambiguous response (~0.5) is set as 1, and the similarity between two compounds responding with values close to 0 or two compounds responding with values close to 1 approaches 0:

$${D}_{2} = \frac{( - {\sum }_{i = 1}^{n} {p}_{i} log {q}_{i} - {\sum }_{i = 1}^{n} {q}_{i} log {p}_{i} )}{2 n log \frac{1}{2}}$$

A6. A display according to A1, wherein the similarity between the compounds is computed using AB-divergence formulated as:

$${D}_{AB}^{( α , β )} = - \frac{1}{α β} {\sum }_{i = 1}^{n} ( {p}_{i}^{α} {q}_{i}^{β} - \frac{α}{α + β} {p}_{i}^{α + β} - \frac{β}{α + β} {q}_{i}^{α + β} )$$

where α and β are the parameters of the divergence.

A7. A display according to A1, wherein the similarity between the compounds is computed using AB-divergence formulated alternatively as:

$${D}_{AB}^{( α , β )} = {\sum }_{i = 1}^{n} {d}_{AB}^{( α , β )} ( {p}_{i} , {q}_{i} )$$

$$where$$

$${d}_{AB}^{( α , β )} ( {p}_{i} , {q}_{i} ) = \{ \text{} \begin{matrix}- \frac{1}{α β} ( {p}_{i}^{α} {q}_{i}^{β} - \frac{α}{( α + β )} {p}_{i}^{α + β} - \frac{β}{( α + β )} {q}_{i}^{α + β} ) & for α , β , α + β \neq 0 \\ \frac{1}{{α}^{2}} ( {p}_{i}^{α} ln \frac{{p}_{i}^{α}}{{q}_{i}^{α}} - {p}_{i}^{α} + {q}_{i}^{α} ) & for α \neq 0 , β = 0 \\ \frac{1}{{α}^{2}} ( ln \frac{{q}_{i}^{α}}{{p}_{i}^{α}} + {( \frac{{q}_{i}^{α}}{{p}_{i}^{α}} )}^{- 1} - 1 ) & for α = - β \neq 0 \\ \frac{1}{{β}^{2}} ( {q}_{i}^{β} ln \frac{{q}_{i}^{β}}{{p}_{i}^{β}} - {q}_{i}^{β} + {p}_{i}^{β} ) & for α = 0 , β \neq 0 \\ \frac{1}{2} {( ln {p}_{i} - ln {q}_{i} )}^{2} & for α , β = 0\end{matrix} . \text{}$$

where p and q are the vectors describing the compounds.

B1. A system for enhancing human-computer interaction, comprising:
(A) One or more selectable databases of information on a plurality of chemical compounds wherein the information in the one or more selectable databases alone or in combination comprises chemical, physical and structural information and biological information for each compound;
(B) A computational device to calculate for compounds selected from one or more of the selectable databases a relative location in a biological parameter space for each of the selected compounds based on biological information for the compound in the database;
(C) A computational device to calculate for compounds selected from one or more of the selectable databases a relative location in a chemical, physical and/or structural properties parameter space for each of the selected compounds based on chemical, physical and/or structural information for the compound in the database;
(D) A computational device to display the parameter spaces of (B) and (C) and to map the selected compounds therein;
(E) One or more computational modules for human-computer interaction effective for users to:
   (a) select databases from which information is to be displayed;
   (b) select compounds to display from each selected database;
   (c) select biological, physical, chemical and/or structural parameter spaces in which compounds will be represented, including spaces based on combinations of the parameters;
   (d) select methods for calculating the location and/or distance of compounds in the parameter spaces;
   (e) adjust parameters of the parameter space, including scales;
   (f) select and display additional information about compounds;

B2. A system according to B1, where the location of compounds in the biological parameter space is determined by one or more dissimilarity and/or a similarity metrics.

B3. A system according to any one of B1 to B2, wherein the metric is the Cell Health Index.

B4. A system according to any one of B1 to B3, wherein biological assay measures are stretched between 0 and 1, where 0 denotes lack of response, and 1 denotes maximal observable response, and the similarity between responses is constructed as a distance between these bound response vectors

$${D}_{1} = {( {\sum }_{i = 1}^{n} {( log ( \frac{1}{1 - {p}_{i}} ) + log ( {p}_{i} ) - log ( \frac{1}{1 - {q}_{i}} ) - log ( {q}_{i} ) )}^{2} )}^{\frac{1}{2}}$$

where p and q are the vectors describing the compounds.

B5. A system according to any one of B1 to B4, wherein the similarity between two compounds that demonstrate ambiguous response (~0.5) is set as 1, and the similarity between two compounds responding with values close to 0 or two compounds responding with values close to 1 approaches 0:

$${D}_{2} = \frac{( - {\sum }_{i = 1}^{n} {p}_{i} log {q}_{i} - {\sum }_{i = 1}^{n} {q}_{i} log {p}_{i} )}{2 n log \frac{1}{2}}$$

B6. A system according to any one of B1 to B4, wherein the similarity between the compounds is computed using AB-divergence formulated as:

$${D}_{AB}^{( α , β )} = - \frac{1}{α β} {\sum }_{i = 1}^{n} ( {p}_{i}^{α} {q}_{i}^{β} - \frac{α}{α + β} {p}_{i}^{α + β} - \frac{β}{α + β} {q}_{i}^{α + β} )$$

where α and β are the parameters of the divergence.

B7. A display according to A1, wherein the similarity between the compounds is computed using AB-divergence formulated alternatively as:

$${D}_{AB}^{( α , β )} = {\sum }_{i = 1}^{n} {d}_{AB}^{( α , β )} ( {p}_{i} , {q}_{i} )$$

$$where$$

$${d}_{AB}^{( α , β )} ( {p}_{i} , {q}_{i} ) = \{ \text{} \begin{matrix}- \frac{1}{α β} ( {p}_{i}^{α} {q}_{i}^{β} - \frac{α}{( α + β )} {p}_{i}^{α + β} - \frac{β}{( α + β )} {q}_{i}^{α + β} ) & for α , β , α + β \neq 0 \\ \frac{1}{{α}^{2}} ( {p}_{i}^{α} ln \frac{{p}_{i}^{α}}{{q}_{i}^{α}} - {p}_{i}^{α} + {q}_{i}^{α} ) & for α \neq 0 , β = 0 \\ \frac{1}{{α}^{2}} ( ln \frac{{q}_{i}^{α}}{{p}_{i}^{α}} + {( \frac{{q}_{i}^{α}}{{p}_{i}^{α}} )}^{- 1} - 1 ) & for α = - β \neq 0 \\ \frac{1}{{β}^{2}} ( {q}_{i}^{β} ln \frac{{q}_{i}^{β}}{{p}_{i}^{β}} - {q}_{i}^{β} + {p}_{i}^{β} ) & for α = 0 , β \neq 0 \\ \frac{1}{2} {( ln {p}_{i} - ln {q}_{i} )}^{2} & for α , β = 0\end{matrix} .$$

and where p and q are the vectors describing the compounds.

C1. A display and/or system according to any of the foregoing or the following enumerated paragraphs in which the visualization and spatial grouping, arrangement or clustering of chemical structures is based on differences and/or similarities in one or more structural or biological features.

C2. A display and/or system according to any of the foregoing or following enumerated paragraphs in which one or more physical chemical properties for each chemical compound is combined with one or more biological parameters that are derived from each compound that describe the effect of each compound on intracellular, extracellular or biological proteins, processes or pathways within a cell.

C3. A display and/or system according to any of the foregoing or following enumerated paragraphs in which one or more chemical substructures is combined with one or more biological parameters that are derived from the effect of a chemical containing those substructures, on intracellular, extracellular or biological proteins, processes or pathways within one or more cells is displayed.

C4. A display and/or system according to any of the foregoing or following enumerated paragraphs that visualizes and/or displays one or more biological parameters for a compound that are derived from the effect of the chemical containing those substructures on intracellular, extracellular, or biological proteins, processes or pathways within one or more cells.

C5. A display and/or system according to any of the foregoing or following enumerated paragraphs that visualizes and/or displays one or more acute cellular stress parameters that combine mitochondrial effects with cellular stress effects.

C7. A display and/or system according to any of the foregoing or following enumerated paragraphs that visualizes and/or displays one or more parameters that are associated with inflammation pathways.

C8. A display and/or system according to any of the foregoing or following enumerated paragraphs that visualizes and/or displays one or more parameters that are associated with any one or more of DNA Damage Repair, epigenetic regulation, cell cycle and/or GPCR binding and downstream signaling within cells.

C9. A display and/or system according to any of the foregoing or following enumerated paragraphs that visualizes and/or displays one or more parameters measured in whole organism screens such as screens in any one or more model organisms, including but not limited to zebrafish C. elegans, Daphina, mice, rats, Drosophila, among others, and including behavioral and phenotypic parameters, as well as biological and biochemical parameters.

C10. A display and/or system according to any of the foregoing or following enumerated paragraphs that visualizes and/or displays one or more parameters that define changes to the morphology of a cell, either direct measurements or calculated measurements that define changes to internal cellular complexity, function, or shape.

C11. A display and/or system according to any of the foregoing or following enumerated paragraphs that visualizes and/or displays one or more parameters that are collectively termed “cell painting”.

D1. A display and/or system according to any of the foregoing or following enumerated paragraphs that visualizes and/or displays biological parameters combined with physical-chemical properties and/or chemical structures.

E1. A display and/or system according to any of the foregoing or following enumerated paragraphs that visually highlights the spatial position of a new, unknown compound within a “universe” of other compounds visualized based on the same chemical or biological features, wherein the visualization groups compounds (distance function) based on calculated similarity and/or difference “distances” between compounds based on the relationship between properties associated with the chemical structure and the effect of that chemical structure on one or more biological processes, pathways or proteins.

F1. A display and/or system according to any of the foregoing or the following enumerated paragraphs utilizing virtual reality (VR) hardware, which may include, but is not limited to, standalone VR headsets, PC-connected VR headsets, gaming console VR headsets, mixed-reality and enhanced reality headsets, mobile VR headsets, motion controllers and motion platforms, and haptic feedback devices, such as gloves, suits, and vests that provide tactile feedback.

F2. A display and/or system according to any of the foregoing or the following enumerated paragraphs, wherein a compound with a position represented within the universe based on any of the foregoing enumerated paragraphs can be captured, separated or removed away from, or outside of, the “universe”, network or cluster of compounds, using physical human motion encoded capturing tools provided by and within the VR hardware and software.

F3 A display and/or system according to any of the foregoing or the following enumerated paragraphs, wherein the full compound network or “universe”, with compound positions represented within the universe based on any of the foregoing enumerated paragraphs, can be rotated to expose/reveal, visualize and zoom in to different clusters of compounds, using physical human motion encoded capturing tools provided by and within the VR hardware and software.

F4 A display and/or system according to any of the foregoing or the following enumerated paragraphs, wherein biological results or physical chemical properties for any isolated or targeted compounds within a network can be displayed above the compound node in 3D space based on the tools provided by and within the VR hardware and software.

F5 A display and/or system according to any of the foregoing or the following, enumerated paragraphs, wherein a VR allows the user to point a virtual laser at a node (in embodiments a compound represented in space based on its biological/chemical/or combination thereof similarity or dissimilarity). “grab” that compound and move it away from or outside of a condensed cluster of other items (compounds) to facilitate review or visualization of detailed information about the selected item.

F6. A system according to any of the foregoing or the following enumerated paragraphs, comprising a virtual reality (VR) display wherein information is displayed and can be manipulated in a virtual reality environment.

F7. A system according to any of the foregoing or the following enumerated paragraphs, wherein the VR display comprises one or more of the a standalone VR headset, a PC-connected VR headset, a gaming console VR headset, a mixed-reality headset, an enhanced reality headset, a mobile VR headset, a motion controller, a motion platform, a haptic feedback device.

F8. A system according to any of the foregoing of the following enumerated paragraphs, wherein the haptic feedback device is any one or more of the following that provide tactile feedback to users: haptic feedback gloves, haptic feedback suits, and haptic feedback vests.

F9. A system according to any of the foregoing or the following enumerated paragraphs, wherein compounds are represented as distinct locations in a multiparametric parameter space defined by physical, chemical or biological parameters or combinations thereof, wherein individual compounds can be selected, captured, moved and separated from other compounds, and as desired to outside the parameter space, using physical human motion encoded capturing tools provided by and within the VR subsystem of hardware and software.

F10. A system according to any of the foregoing or the following enumerated paragraphs, wherein one or more pluralities of compounds within a parameter space can be selected and then manipulated without affecting other compounds in the parameter space.

F11. A system according to any of the foregoing or the following enumerated paragraphs, wherein such pluralities can be rotated, enlarged, made smaller and otherwise visually manipulated using physical human motion encoded capturing tools provided by and within the VR hardware and software.

F12. A according to any of the foregoing or the following enumerated paragraphs, wherein physical, chemical and biological characteristic for a selected compound can be displayed in the multiparameter space using tools provided by and within the VR hardware and software.

### BRIEF DESCRIPTION OF THE FIGURES

FIG. 1 shows a schematic chart of an embodiment of inventions herein described using biological similarity space for chemical data visualization. Step 1—Gather a set of biologically-active chemical compounds (including a set of known controls). Step 2—Measure the action of each compound in phenotypic screens S1 and calculate a phenotypic response vector for each compound in the screen. Step 3—Merge the data from the screens via data concatenation, or a fusion of distance matrices from each of the screens, created with an appropriate distance metric. Step 4—Display a 3D network with biochemical/chemical compounds represented as nodes (vertices) and (dis)similarities in generated biological phenotypes represented as edges. Step 5—Annotate the vertices with external data. Repeat Steps 1 through 5 for n additional compounds. e.g., execute phenotypic screen S2 to measure the action of compound in the phenotypic screen and calculate a response vector 2; etc. up to compound n. Step 6—Display the resulting data in a 3D (or multidimensional) space/representation, wherein the display can be in an Euclidean (for instance, via multidimensional scaling) or non-Euclidean geometry (i.e., manifold learning). Step 7—Interactive interrogation of the compounds in the biological space, including structural information from chemical and bio-biochemical compound databases

FIG. 2 illustrates general aspects of the implementation of the display systems described herein. As the Figure shows, information on structures and biological activities is Stored in a database, and properties are calculated thereon. The information can be Organized as a user desires into fields and tables, for instance, and data from multiple sources can be standardized. The data is Analyzed using a variety of tools, particularly to calculate distances in biological parameter spaces. The results of the analysis are Visualized.

FIG. 3 is a screenshot illustrating a display in accordance with embodiments herein described in which each compound is displayed as a dot (sphere) in a selected biological parameter space. The dots representing the compounds show as a cluster (or cloud). The distances between the dots in the cloud scale with the difference between the compounds in the biological properties and are used to define the biological parameter space of the display. The Figure shows that individual compounds can be selected, and the display will show information about the selection, such as the chemical structure at the upper right in the Figure. Any other information in the selected database can be called up as well. The figure shows an illustrative menu bar through which the user can select such aspects of the display as whether to display structure similarity or biological similarity.

In the particular embodiment shown in this screenshot, the compound universe network map is based on the similarity of the biological fingerprints in the AsedaSciences screen. The corresponding chemical structure appears in the top right corner when the mouse moves over individual nodes. The shape of this universe is based on the similarity or difference between fingerprints relating to the compound's physical, structural, chemical, or biological characteristics.

FIG. 4 is a screenshot illustrating the application of an expansion tool to the same screenshot illustrated in FIG. 3. The tools allow the user to expand a part of a compound universe. Other tools, to mention just a couple, allow the user to rotate and move the displayed compound universe or pats thereof and to zoom in and out of the display.

In the particular embodiment shown in this screenshot, the compound universe based on the biological fingerprints is separated further based on a tool integrated to allow the network plot to be continually expanded in 3D to expose compounds that might be lying underneath each other in 2D but can be easily seen as a separate cluster when separated away by using the expansion button (red arrow above

FIG. 5 illustrates a display of the chemical similarity of the same compounds displayed in FIGS. 3 and 4. The chemical and biological properties clearly differ greatly.

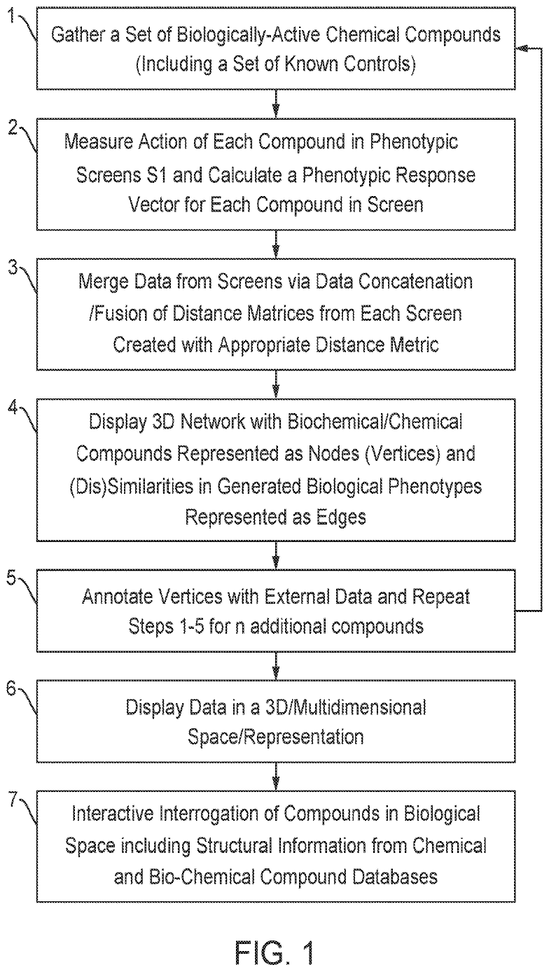

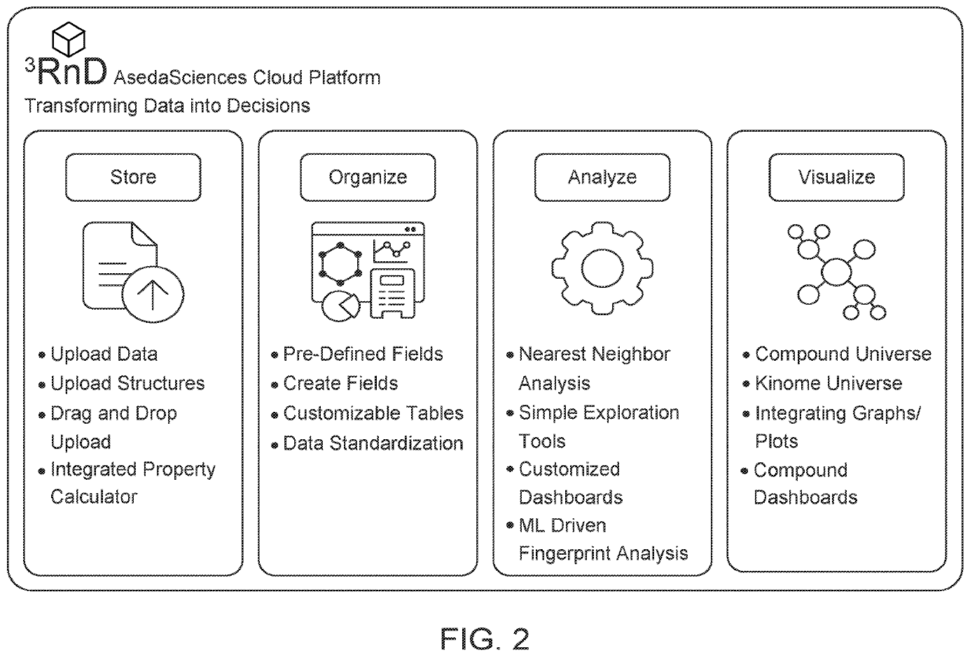

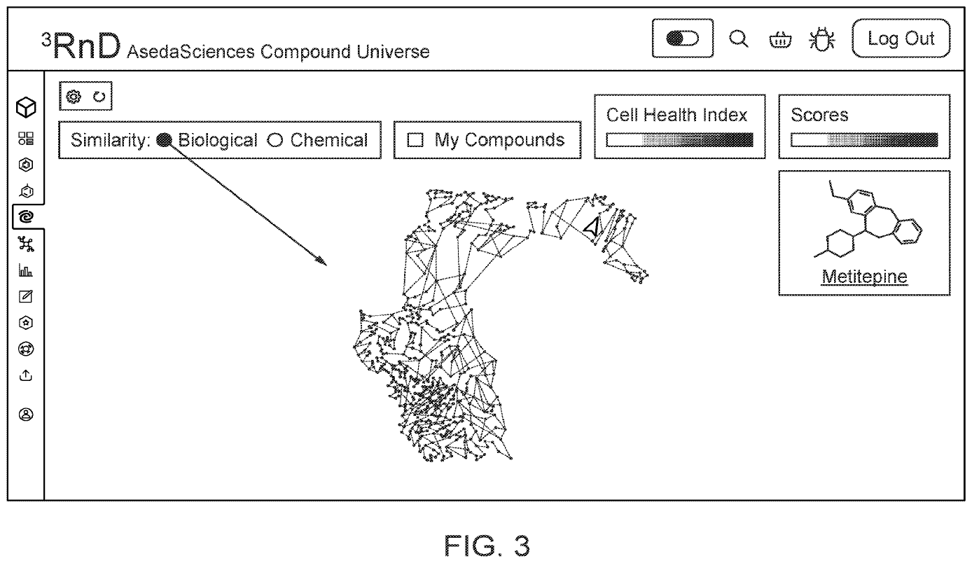

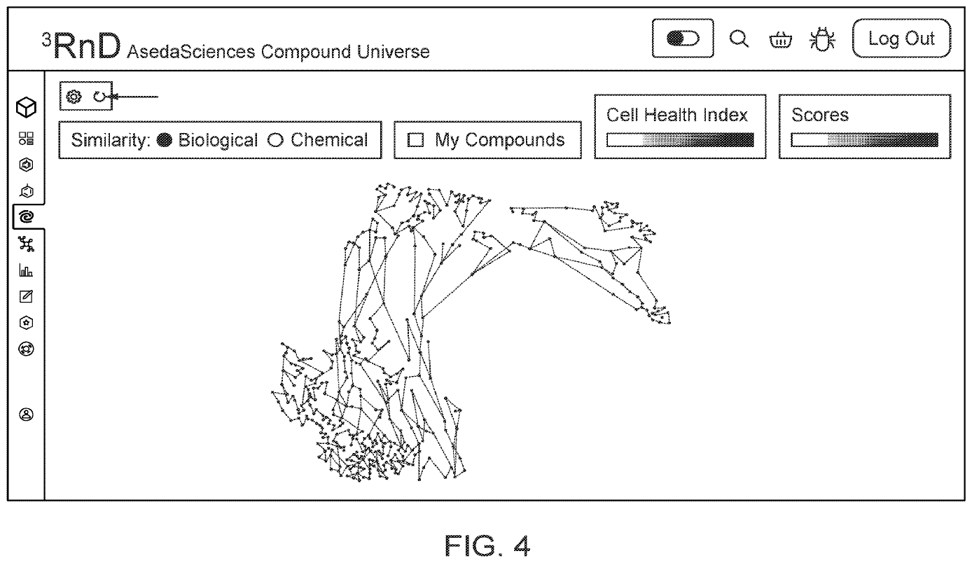

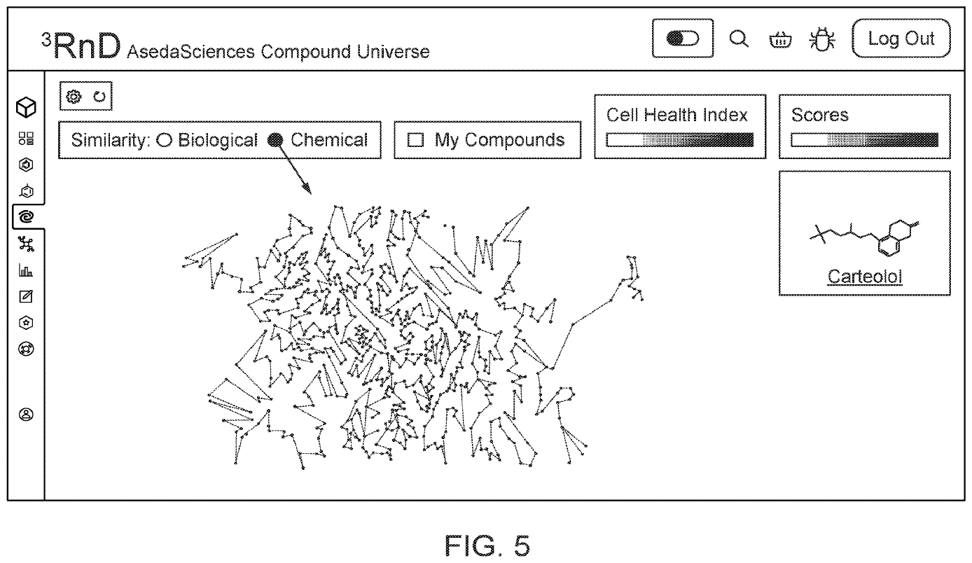

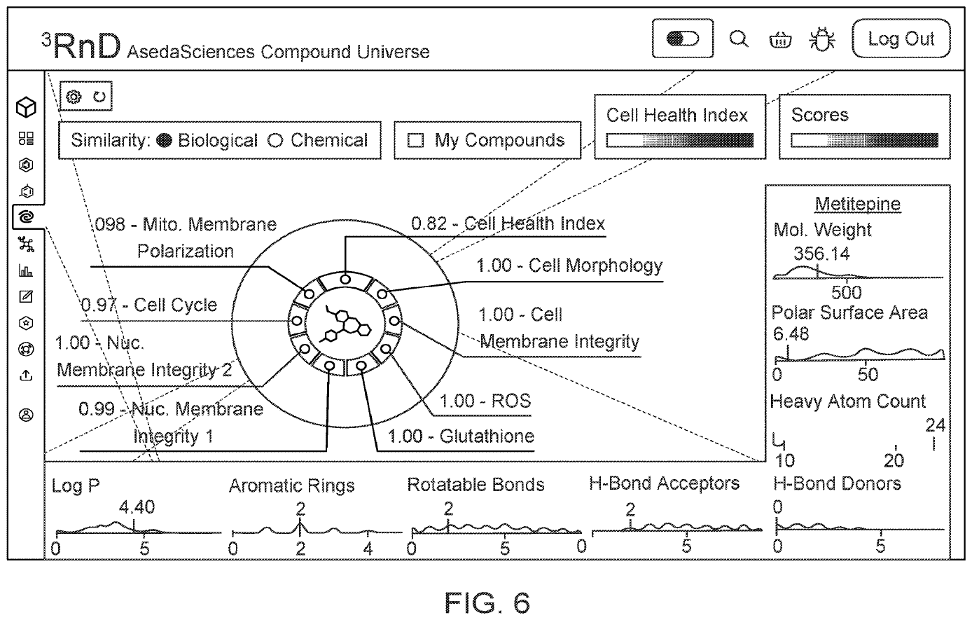

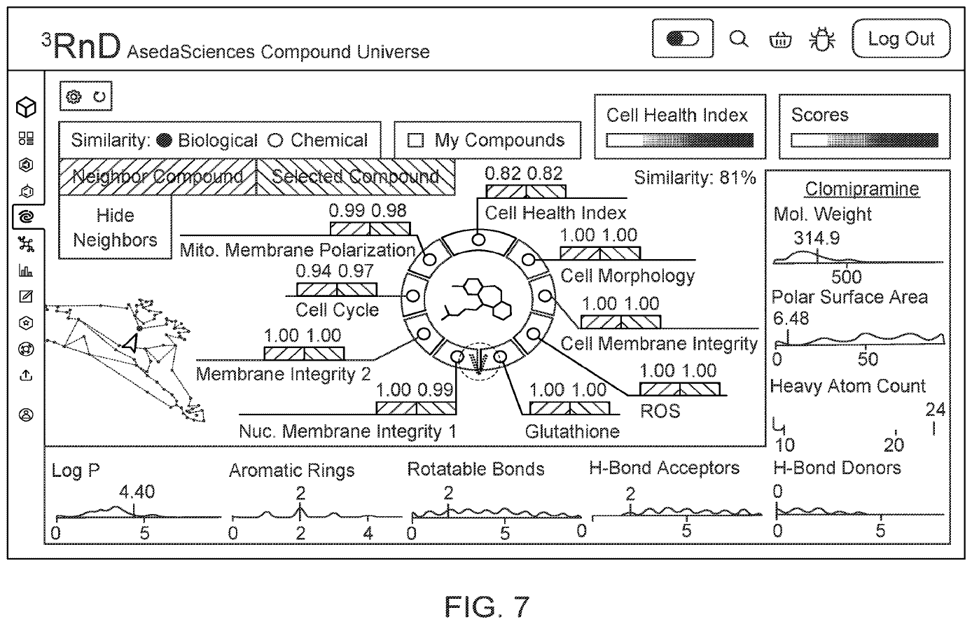

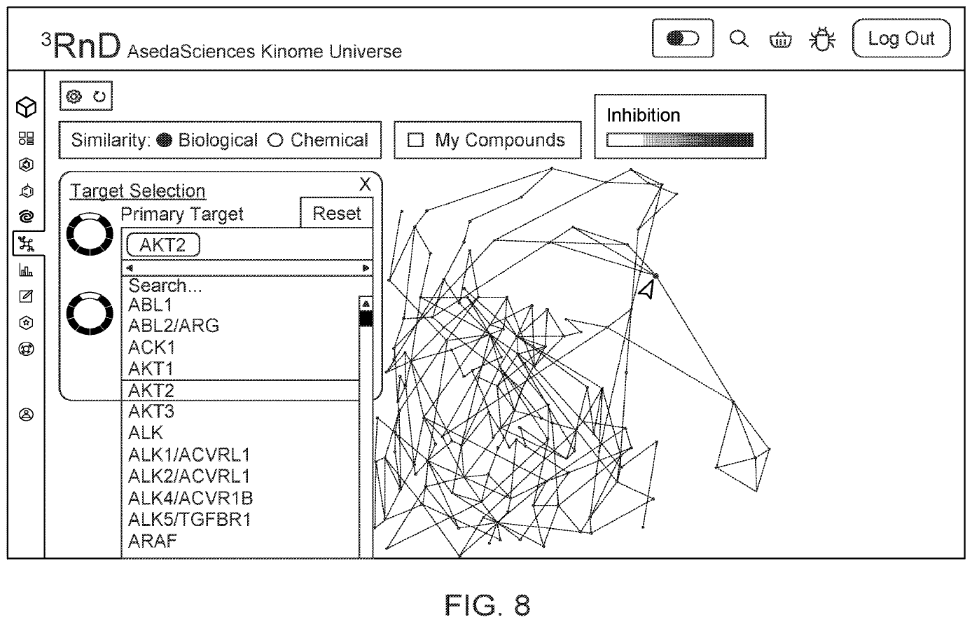

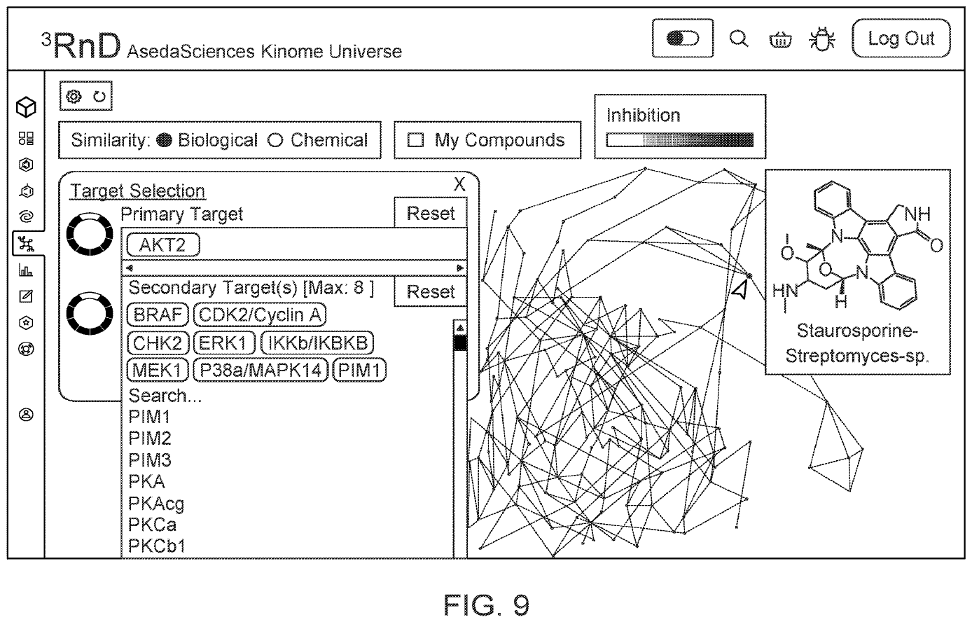

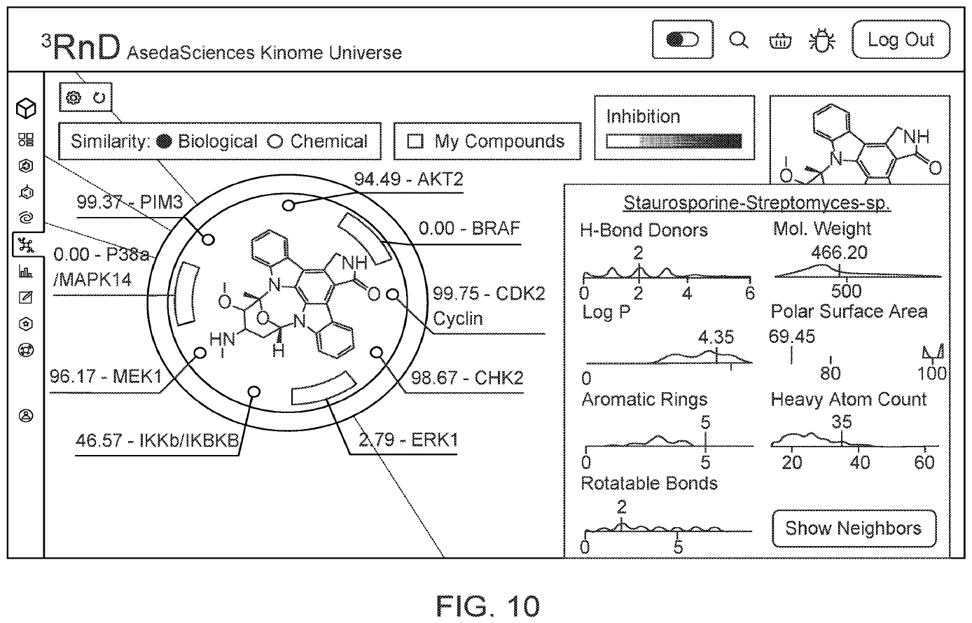

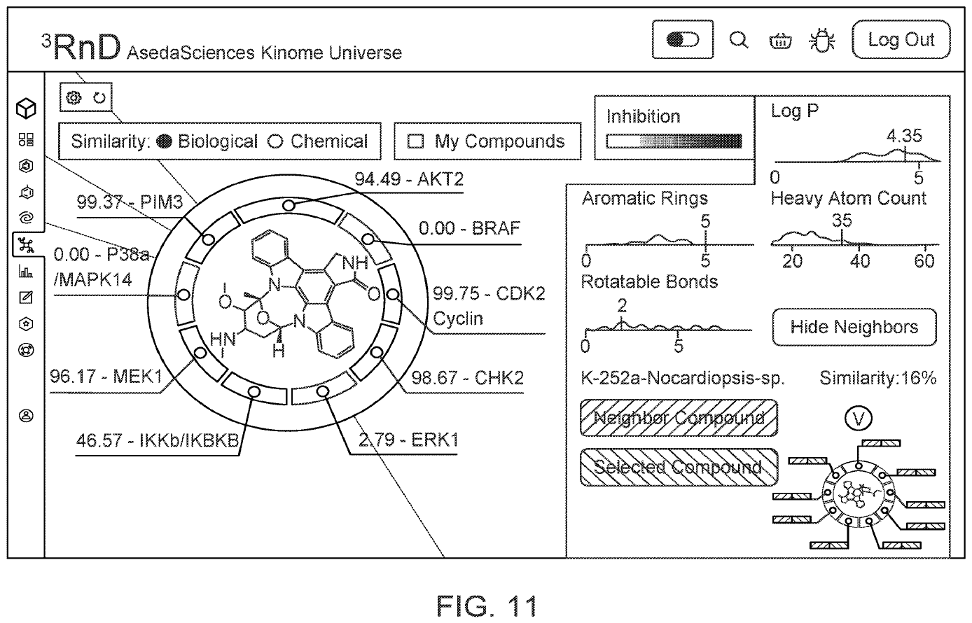

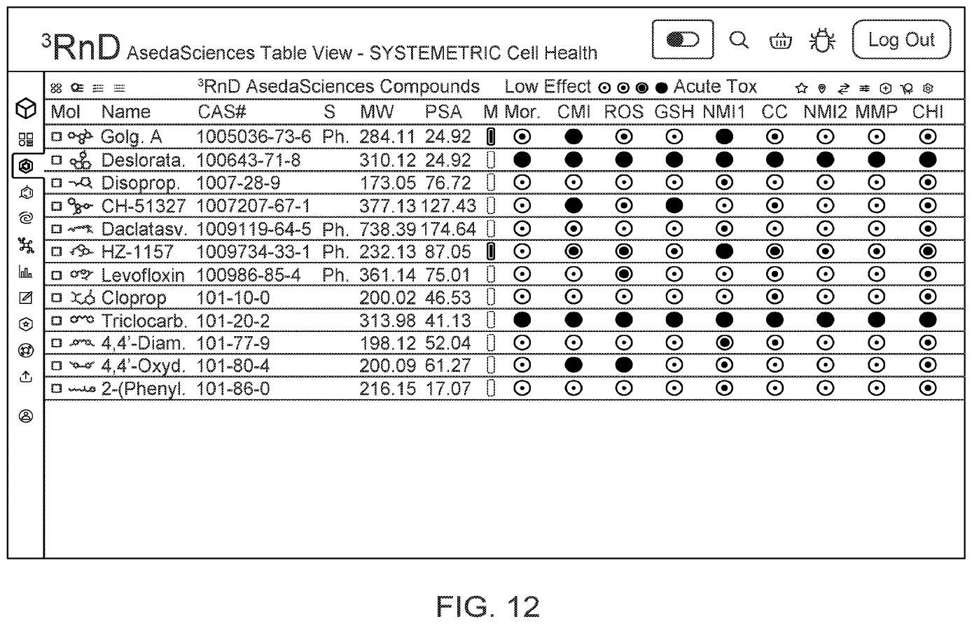

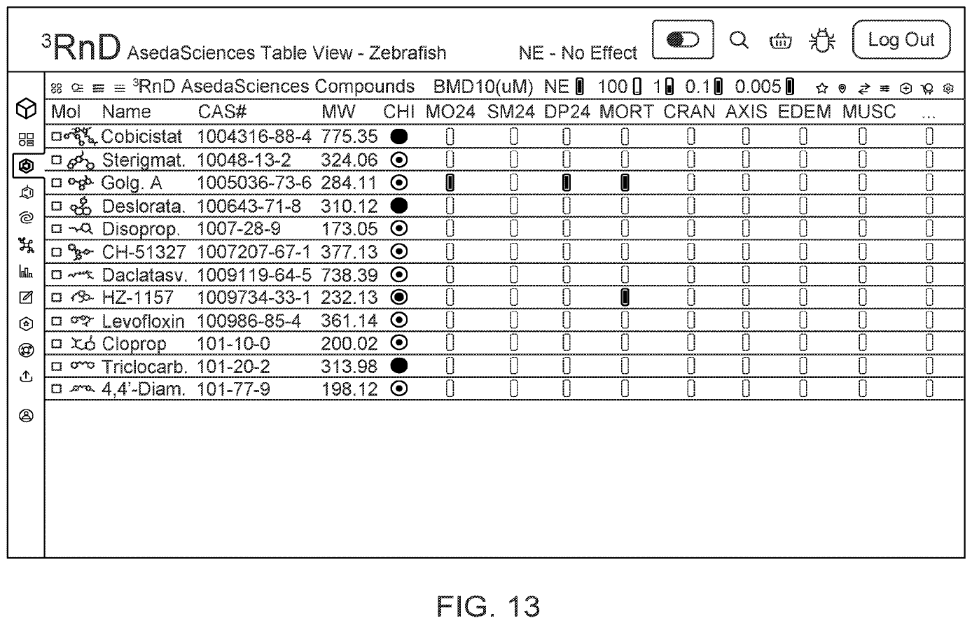

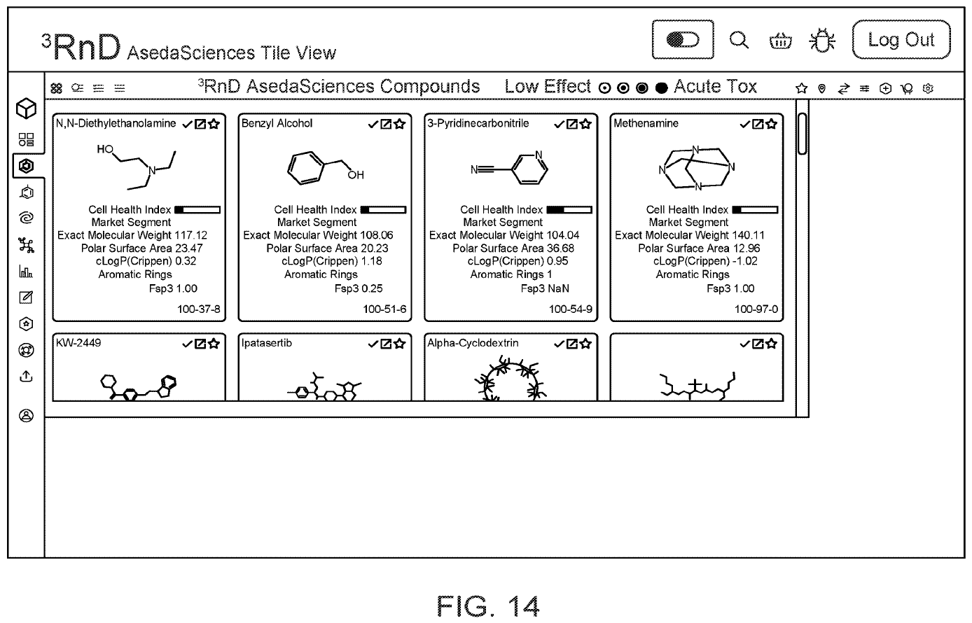

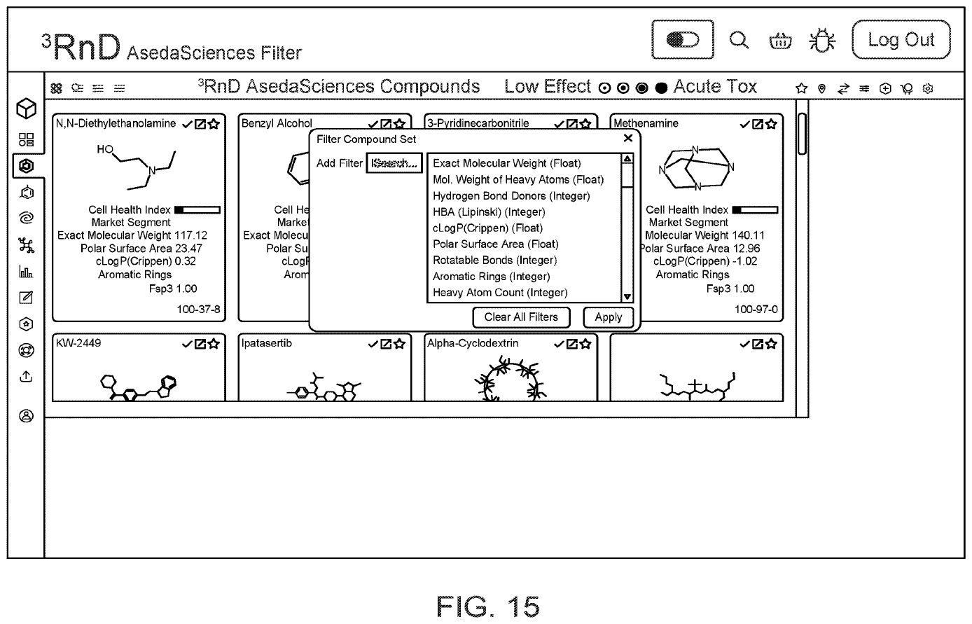

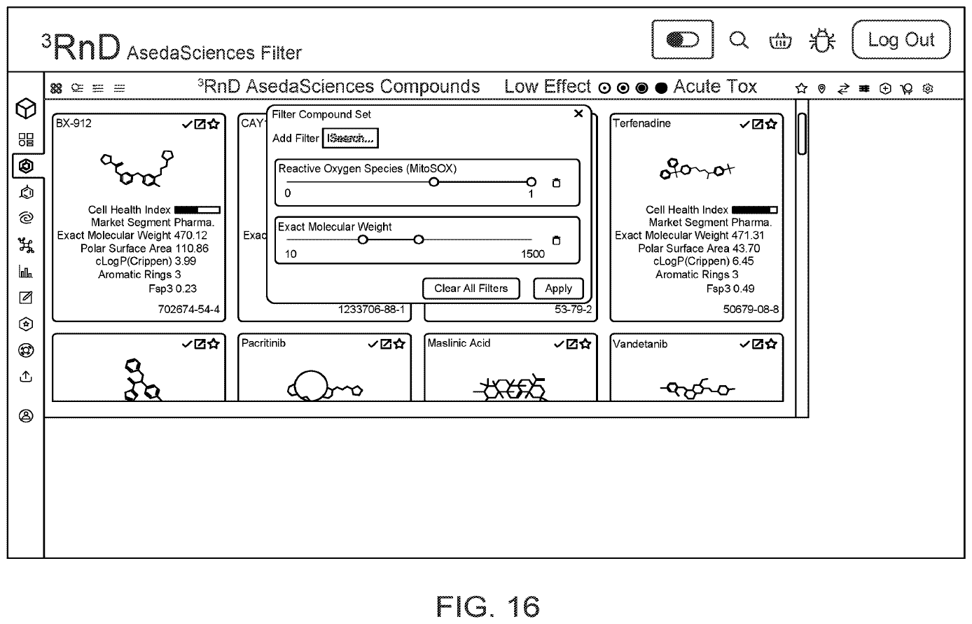

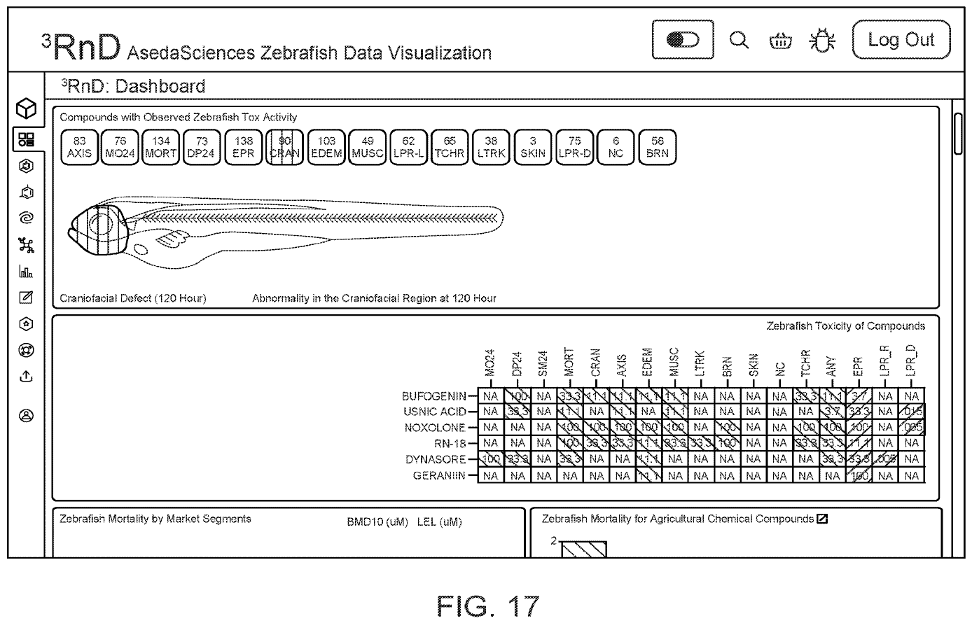

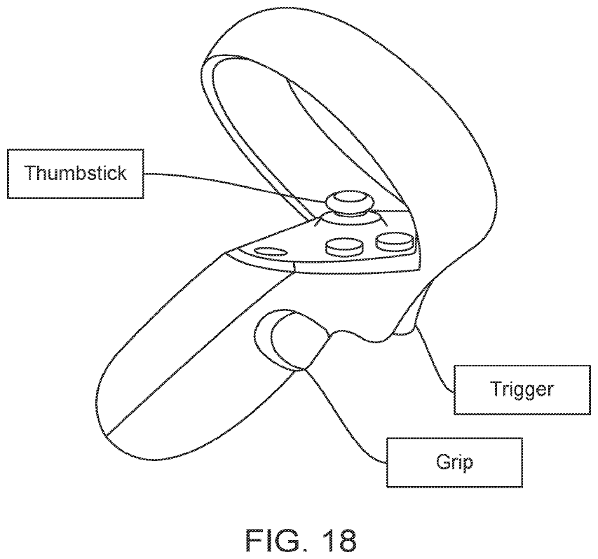

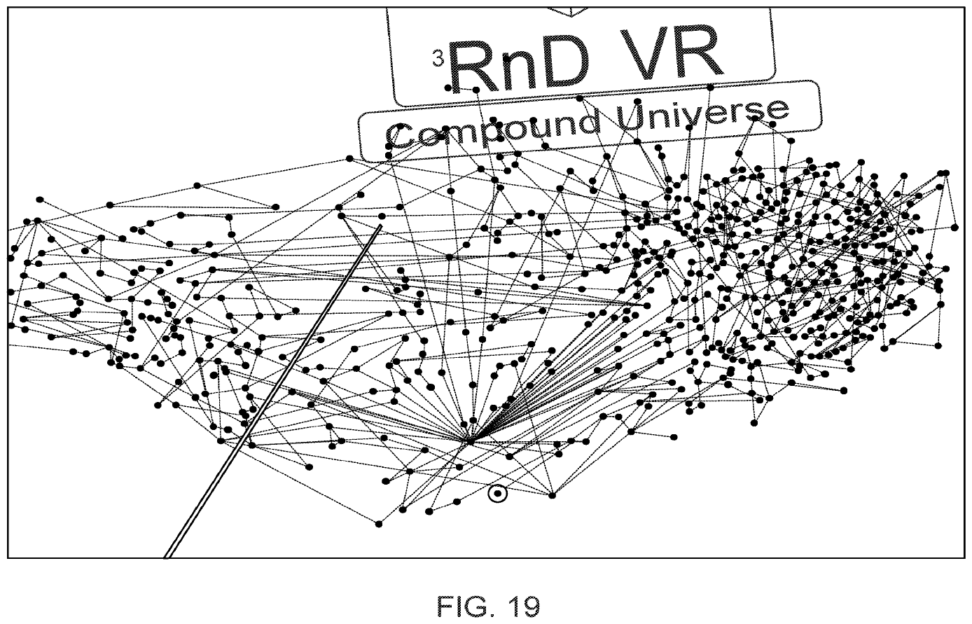

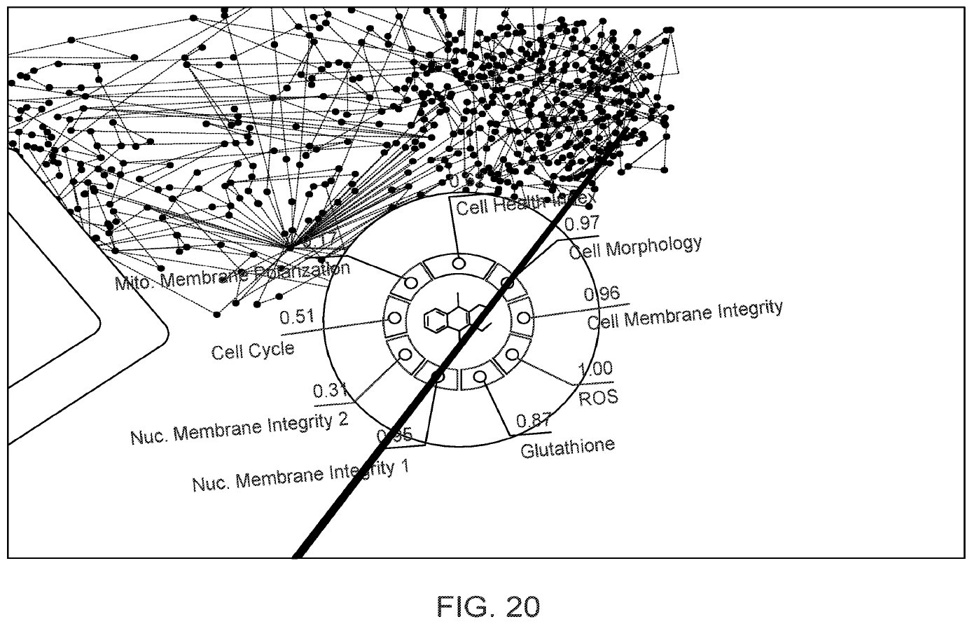

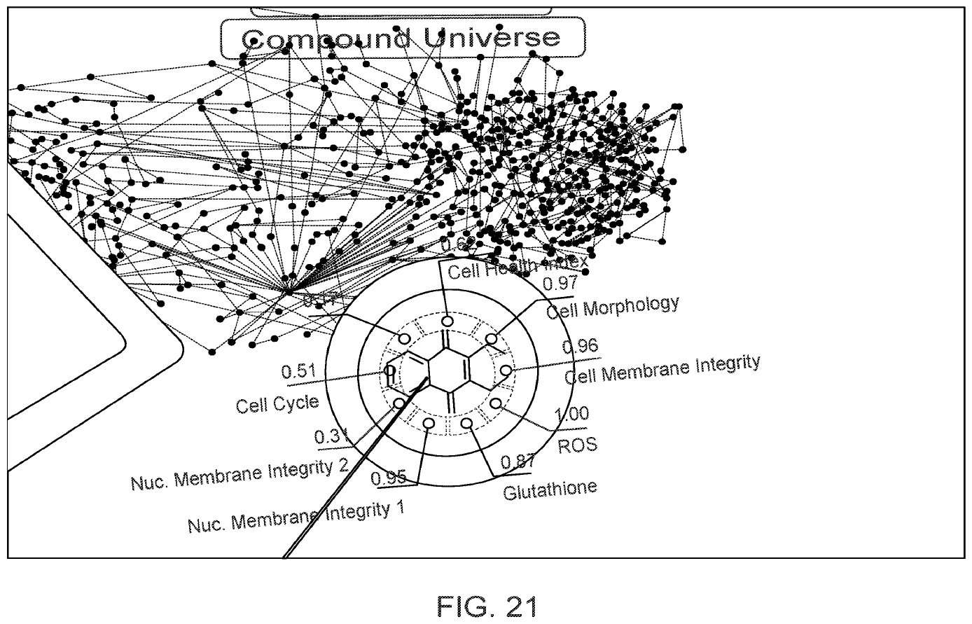

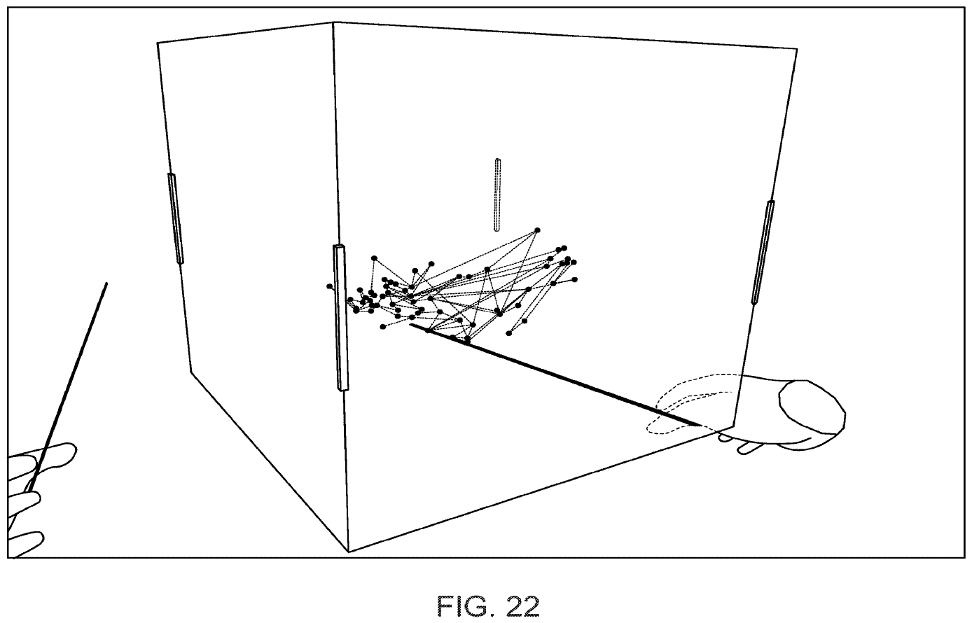

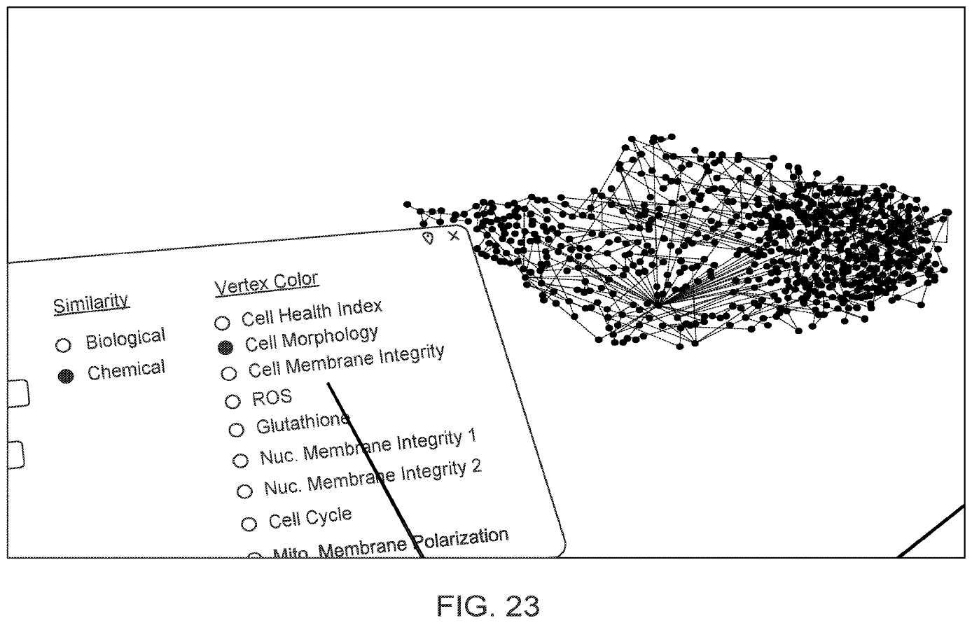

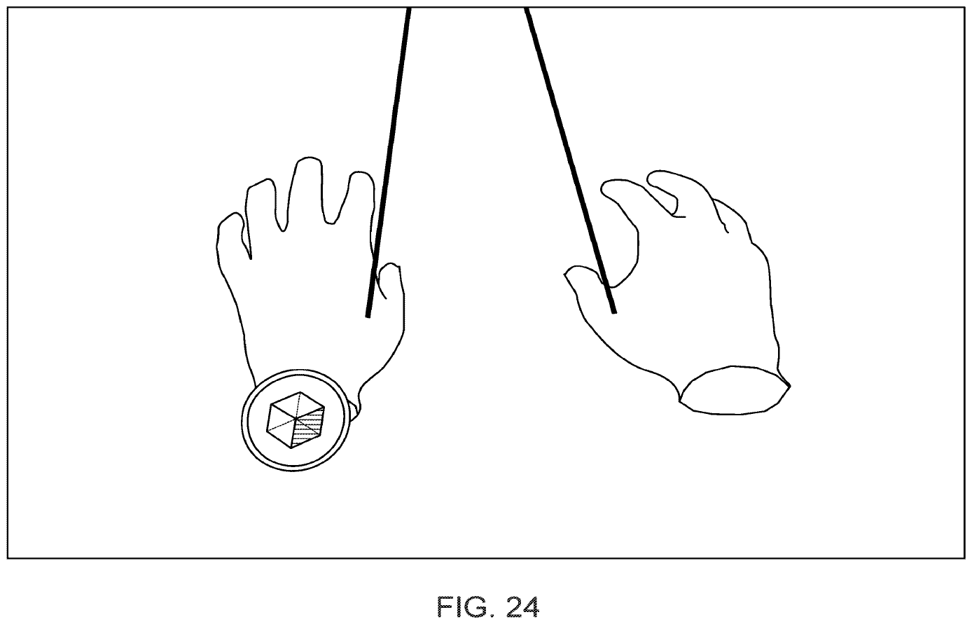

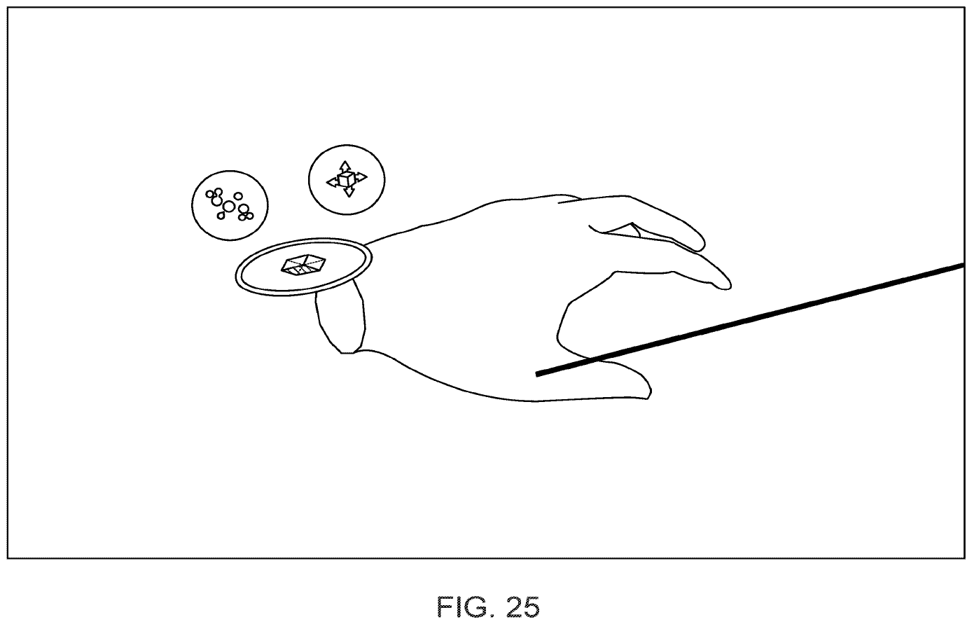

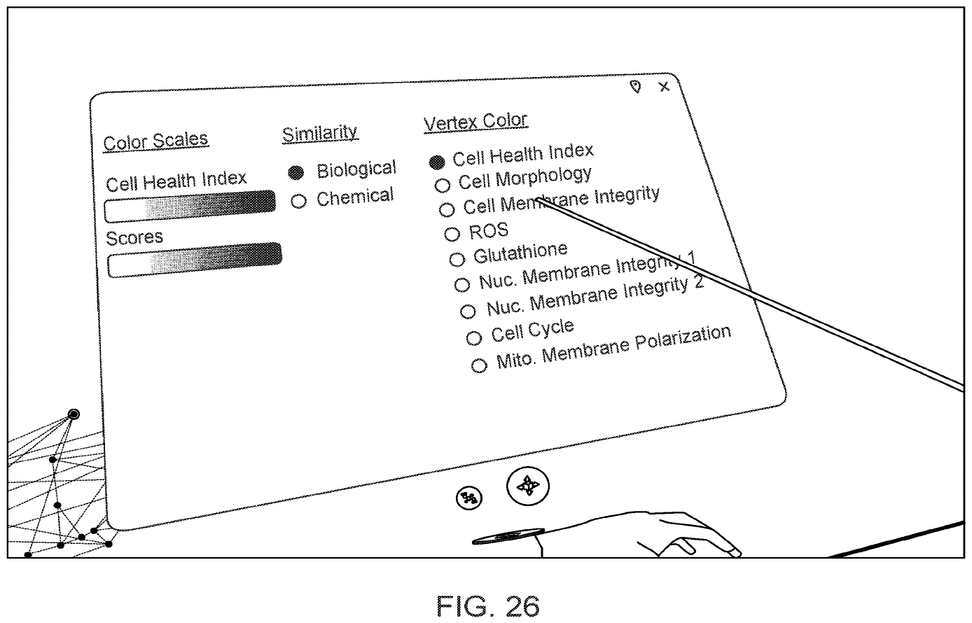

The particular embodiment displayed in this figure shows the same universe of compounds as the previous figure but is arranged based on the similarity of structure. The colors of the nodes represent the score in the Cell Health Index (CHI), derived by a Machine Learning (ML) algorithm from the screen, but could be based on any index or score based on ML derived analysis of multi-dimensional, biological or cellular phenotypic screening data.

This differs substantially from traditional way s of making decisions on chemical structure selection because the system not only displays similarities and differences in chemical structures (and other physical properties) but also displays differences (and similarities), such as toxicity in the figures, wherein the color coding of each node is based on results of a biological toxicity screen. The system and display thus provides the discovery team with information connecting compounds not just by structural, physical, and chemical properties but also, by their biological properties. This is particularly powerful for highly multi-dimensional biological, physical and chemical datasets. Displays such as this enable discovery efforts to more effectively visualize compounds by biological similarity and difference, aiding lead compound selection, assessing the impact of scaffold modifications during SAR series development, developing strategies for exploring chemical space to identify promising drug candidates, and ruling out compounds by likelihood of off-target and harmful side effects. For example, lead compound selections can represent the differences in chemicals based on structure but are color-coded for their biological properties.

Displays in accordance with some embodiments are thus useful for prioritizing and selecting compounds within a structure-activity relationship series by showing not only clustering because of chemical similarity but also the different clustering of the compound based on their biological effect. This is shown clearly by the differences in the shapes of the network arrangement based on chemical structures in this figure versus the clustering by biological properties shown in the previous figure.

FIG. 6 illustrates a display of additional information on the biological properties of one of the compounds in a display. When a node is selected, additional information about the corresponding compound is displayed. In the illustrated embodiment, biological characteristics are displayed around the outside of a type of pie chart, making it simpler to visually understand the risk associated with the compound. At the same time, the position of this compound's physical-chemical properties is displayed relative to the entire database of compounds at the right. Each node can have one or multiple rings of segments that define different biological results or physical chemical properties of a compound for ease of differentiation and comparison, allowing similarity analysis based on the color coding of each individual and combinations of segments.

While biological properties in this figure are displayed in the form of a pie chart, many other forms of display are possible. The pie chart in this particular illustration shows nine different biological activities for the compound, namely effects of the compound on Mitochondrial membrane polarization; the cell cycle, nuclear membrane integrity by measure 1; nuclear membrane integrity by a second measure; glutathione level; ROS, cell membrane integrity; cell morphology; and Cell Health Index. At the same time, the physical properties of the compound are displayed in the box on the right in the screenshot, effectuating a better understanding of the biological effects, positive and negative, of the compound in the context of its physical properties (in this case risk assessed by a toxicity screen).

FIG. 7 illustrates a screenshot of a display option in which the compound cluster is displayed with the cursor arrow pointing to a selected compound and the biological and physical properties of a near neighbor of the compound displayed in the two boxes to the right.

The previous figures illustrate the display of nearest neighbor compounds based on their similarity of the biological fingerprints while showing the similarity in chemical structure. Nearest neighbor chemicals importantly can be viewed and compared based on the biological characteristics (results of biological screens), shown in the figure as bar graph plots around the circle, alongside physical-chemical properties (histograms above) simultaneously in a graphical manner.

This feature greatly facilitates the selection of compounds based on the combination of biological and physical parameters. In one application, this facilitates and reduces risk in selecting compounds within a structure-activity relationship series that may only vary by a single side chain around a scaffold. For instance, these displays, in accordance with the invention, facilitate the selection of side chains that are safer while enhancing drug-like properties that are based on other parameters.

FIG. 8 is a screenshot illustrating a display of compound binding to AKT2. As illustrated, a plurality of kinases are listed in a pull-down menu and can be selected by a user to display the binding of compounds in a database thereto. Compounds can be individually selected to display biological and chemical physical and structural properties, as illustrated in the foregoing figures.

In particular, the figure shows a Kinome Universe, which uses results from a kinome screen that measures the activity of a kinase inhibitor against a multitude of kinases (e.g., 300 kinases). The display can show such things as binding to a recombinant protein to measure inhibition of a probe or inhibition of function in vitro, cell-based, and/or in vivo assays.

The display is agnostic to the way that impact on a kinase is measured. It represents the chemical structures based on their effects on measurements against one or more kinases. Any number can be used. In much the same way as for this kinome screen, any biological screen can be represented, such as a GPCR screen or a binding screen, of screens for other types of enzymes and receptors.

The compounds here are arranged in the network based on the results from 300 kinases. The pull-down tool allows users to color-code the compounds of interest rapidly and pinpoint those compounds rapidly in the network. In this embodiment, zooming in with the zoom tool reveals chemical structure. The network represents arrangement-based similarity or dissimilarity of the biological effects of the compounds in a screen or in a combination of screens, the results of which have been combined in some way.

FIG. 9 is a screenshot illustrating an elaboration of the screenshot in FIG. 8, wherein the display further reflects binding to several secondary targets, and structural information is displayed for the selected compound.

This screenshot exhibits the ability of the user to choose secondary targets, such as unwanted binding or potential toxicity-related kinases, and to color code the nodes in the network based on that selection, allowing rapid selection of compounds in seconds that would be difficult to do through a spreadsheet.

FIG. 10 is a screenshot illustrating (1) a pie chart showing the binding of the selected compound to nine targets (with numerical indices of binding activity) and (2) physical properties and the structure of the compound.

FIG. 11 is a screenshot similar to FIG. 10 but showing the biological and physical properties of a near neighbor of the compound selected in FIG. 10.

It illustrates that a user can click on a node and zoom in and compare nearest neighbors based on the similarity of biological results in a screen, such as a Kinome screen, a zebrafish screen or any multiparametric biological screen. Traditionally, comparisons are made based on chemical structure and characteristics; here, the network maps are based on biological effects, and they retain the ability to look at the structure and compare physical-chemical properties

FIG. 12 is a screenshot illustrating a type of heat map displaying the “heat” of a series of compounds in a panel of biological “assays” of the Cell Health Screen. This type of user-selectable display provides a comparison of the biological activities of individual compounds in individual in vitro biological assays.

It shows that compounds can be compared together with chemical structure and a visual representation of each biological result. This would traditionally be done purely with numbers, which are difficult to view relative to each other. The screenshot here shows how easily it is to see the extent of risk based on both size and color coding and have different biological results represented simultaneously, but with different shapes and shading that can still show a quantitative representation of toxicity for different types of screens, while still allowing the comparison of key physical-chemical parameters. The bars represent the results of zebrafish screens. The circles show the results of the Cell Health screen.

FIG. 13 is a screenshot illustrating a type of heat map displaying the “heat” of a series of compounds in in vivo zebrafish assays.

It shows an example of a table view of the data: full bars show that the compound was toxic to that zebrafish organ/parameter at the lowest concentrations (highly toxic), whereas bars that are completely empty show the compound was not toxic at the highest concentration measured. Displays of this type provide a very effective and efficient visual way to compare results and make decisions on compound selection, prioritization, and progression. It accelerates the decision process by arranging chemical similarities or dissimilarities based on the visual representation of their biological effects

FIG. 14 is a screenshot illustrating a “tile view” display of four compounds in which their scores on the Cell Health Index are displayed in a horizontal bar chart, and the physicochemical properties of the compounds are displayed as well. The tile view allows the easy comparison of compounds, showing their structure and the results (bar) of biological screens in a visual format that greatly facilitates the evaluation of results and evidence-based compound selection.

FIG. 15 is a screenshot illustrating the same display as FIG. 14, further showing a pull-down menu that allows users to select additional specific properties (“filters” to display for each selected compound.

These filters can be applied to any parameters stored in an accessible database, so compounds with BOTH the desired chemical characteristics AND the biological characteristics of interest can be easily sorted and compared visually. This filter can be used in the table/tile view and in the various Universe views, to mention just two. These views can arrange compounds based on biological results, not just chemical properties, something not shown in this way in other chemical-biological assessment platforms.

FIG. 16 is a screenshot illustrating a user-selectable filter engine that can be applied to limit the display to compounds with properties that fall within a user-selectable range of user-selectable parameters.

Filters can be selected so that certain ranges of both chemical and biological parameters can be chosen and only those compounds viewed. This, again, can be used across the various visualization screens

FIG. 17 is a screenshot illustrating the biological activities of compounds in in vivo zebrafish mortality/toxicity assays. Mortality is indicated by color in the zebrafish diagram at the lower left. The toxicity of compounds is illustrated in the heat map at right.

It visualizes a number of compounds that affect each specific organ measurement, and there is an associated image below visualizing the part of the zebrafish that is selected above. Clicking on a button pulls up a table view of the compounds that had a toxic effect on that organ.

FIG. 18 is a diagram showing a controller for a VR system for use in accordance with some of the inventions and embodiments described herein, such as an Oculus Quest controller. The diagram shows a number of controller features including a Left Thumbstick for moving about in the virtual environment, a Right Thumbstick for rotating in the virtual environment, a Trigger to point to and activate objects in the virtual environment, and a Grip Button to grab objects making first with virtual hands.

This and the following figures show how users can explore, grab, hold and modify the features in a VR visualization of a compound universe.

FIG. 19 shows a pointer being used in a VR implementation to select a compound in a ³RnD VR compound universe rendered in a virtual reality display system.

FIG. 20 shows a pointer being used in a VR implementation to select one compound in a cluster and a control button being used to pull up information on some of the properties of the compound.

FIG. 21 shows how a user can rotate the Network Visualization of a cluster of compounds in a VR implementation.

FIG. 22 shows how a user can interact a VR user interface (UI) to manipulate a network visualization (cluster), showing the user's virtual hands interacting with objects in the UI, placing a ray on an interactable component (white ray), allowing the user to activate various functions associated with the selected component using the buttons and sliders on the controller.

FIG. 23 depicts a white beam selecting an item from a menu in the UI of the VR space. To be able to manipulate and change the parameters of a visualization, users are provided with a wearable controller. Using it users can change various features in the VR. For instance users of a compound universe VR can change the colors of nodes, and the scale, orientation and rotation of a cluster. The controls furthermore allow users to apply filters of various types. In the case of a Wearable UI, such as a watch, users can activate the Wearable UI with the ray of the hand that doesn't have the watch attached. Then menus and options will be displayed over this watch.

FIG. 24 shows a pair of hands in a VR both using pointers (rays) whereby multiple actions are performed at the same time to edit and/or modify the visualization. A UI can be divided by a list of “windows” that display available options when a user presses the wearable UI. For instance, a first menu can be about Network properties, such as color and similarities, and a second is about Network transformations such as scaling and rotations, and about applying filters.

FIG. 25 depicts a virtual hand wearing a virtual wrist controller which has been activated to display two menu options. The one on the left in the figure is to detach the UI from the watch and put it in a fixed position in the environment. The one on the right closes the UI and returns to a selection list (as shown in FIG. 26).

FIG. 26 is a selection list with a variety of functions a user can select

### DESCRIPTION

The invention presented here is inspired by previously reported state-of-the-art chemical space visualization systems, but it employs a fundamentally different method for understanding, modeling, and visualizing the interaction between chemical compounds. The key innovation is the utilization of information regarding the biological responses elicited by the substances of interest. In other words, unlike earlier chemical compound visualization systems, the new one is based on the similarity of phenotypes induced by the exposure of biological systems to chemical compounds. This innovative technology provides a new and novel approach to the visualization of biologically-active chemical compounds and takes advantage of functional cell health assays taught by Rajwa and Shankey (2021), or demonstrated by Bieberich et al. (2021)

The compounds are represented by data vectors obtained by performing multivariate functional biological screening, such as the Cell Heath Screen (CHS). The Cell Health Screen is a multiparametric assay executed on an automated flow cytometry system designed for acute cell stress that employs a panel of fluorescent physiological reporting dyes (Rajwa and Shankey, 2021). Instead of merely generating dose-response curves for all different biological readouts, features are formulated by computing user-defined distance functions between test and control wells. For the purpose of ML machine learning (ML), these feature vectors representing all test chemical compounds are utilized as input to a classification process that applies a logistic regression model or other ML techniques to categorize test compounds in relation to a training set. However, for data visualization, the feature vectors can be used directly without the need for training. In other words, following the execution of the CHS, every tested compound is represented by a vector describing the biological response that a particular cell line exhibits upon exposure to the compound. This biological and functional model contrasts significantly with the chemical representation used to define the chemical space shown in the published work (Borrel et al., 2018; Medina-Franco et al., 2022; Reymond, 2015). In the former case, the data vector describes the chemical properties; thus, compounds are regarded as similar in 3-D (or n-D) space if their structure and physicochemical qualities are similar. The CHS-based n-D visualization, on the other hand, is based on the concept of biological property similarity. Two substances are similar if they elicit a comparable biological reaction in a specific biological assay. It is essential to emphasize that two substances with radically dissimilar chemical structures can cause comparable biological responses. In contrast, two substances with similar structures may have opposite biological effects.

Multiple measures of similarity can be defined so that they are compatible with biological data vectors. Here, we present some of the possible implementations. For instance, if the biological responses are stretched between 0 and 1, where 0 denotes complete lack of response, and 1 denotes upper bound of observable responses, the similarity could be constructed as a distance between these (0,1)-bound response vectors p and q:

$${D}_{1} = {( {\sum }_{i = 1}^{n} {( log ( \frac{1}{1 - {p}_{i}} ) + log ( {p}_{i} ) - log ( \frac{1}{1 - {q}_{i}} ) - log ( {q}_{i} ) )}^{2} )}^{\frac{1}{2}}$$

The similarity/dissimilarity of biological responses may also be expressed incorporating the uncertainty associated with the assay. For instance, the similarity between compounds that demonstrate ambiguous response (~0.5) will be set to reach 1, and the similarity between compounds responding closely to 0 or 1 (for instance, in the toxicology setting, strong toxins or inert compounds) will approach 0. The following scaling of the dissimilarity measure allows for such quantification:

$${D}_{2} = \frac{( - {\sum }_{i = 1}^{n} {p}_{i} log {q}_{i} - {\sum }_{i = 1}^{n} {q}_{i} log {p}_{i} )}{2 n log \frac{1}{2}}$$

Multiple other metrics can be used to define the biological similarity in the space. In principle, the measures of dissimilarity should take into account the distribution of the measured biological properties. Although the preferred embodiment assumes the use of the cell-health assay, the compounds could also be positioned in the space defined by macroscopic biological responses, such as behavioral traits expressed by animals exposed to the presence of the compound. These types of assays might use Zebrafish (Dario rerio), fruit flies (Drosophila melanogaster), nematode worms (Caenorhabditis elegans), or any other well-studied and genetically characterized biological model system. In that case, defining the dissimilarity would involve the knowledge regarding the distributional characteristics of the specific biological metric. For instance, if the distance traveled by a Zebrafish embryo after exposure to light impulse following in the environment with the presence of an investigated substance is used, then the likely statistical model would be based on log-normal or Weibull distributions.

In this and other similar cases, a custom notion of similarity measure may be derived from a general AB-divergence defined as follows (Cichocki et al., 2011; Cichocki and Amari, 2010):

$${D}_{AB}^{( α , β )} = \begin{matrix}\frac{1}{α β} {\sum }_{i = 1}^{n} ( {p}_{i}^{α} {q}_{i}^{β} - \frac{α}{α + β} {p}_{i}^{α + β} - \frac{β}{α + β} {q}_{i}^{α + β} ) \\ for α , β , α + β \neq 0\end{matrix}$$

In limiting cases, with a particular choice of a and 0, the measure above can be reduced to Euclidean distance (α=1, β=1), log Euclidean distance (α→0, β→0). I-divergence (α=1, β=0), Itakura-Saito divergence, and multiple other measures of dissimilarity, for example:

$${D}_{AB}^{( 1 , 0 )} = {\sum }_{i = 1}^{n} ( {p}_{i} log \frac{{p}_{i}}{{q}_{i}} - {p}_{it} + {q}_{i} )$$

$${D}_{AB}^{( 1 , - 1 )} = {\sum }_{i = 1}^{n} ( log \frac{{q}_{i}}{{p}_{i}} + \frac{{p}_{i}}{{q}_{i}} - 1 )$$

$${D}_{AB}^{( 1 , 1 )} = \frac{1}{2} {\sum }_{i = 1}^{n} {( {p}_{i} - {q}_{i} )}^{2}$$

$${D}_{AB}^{( 0 , 0 )} = \frac{1}{2} {\sum }_{i = 1}^{n} {( log {p}_{i} - log {q}_{i} )}^{2}$$

The dissimilarity between biological observation vectors is utilized to generate a dissimilarity matrix, which is then used to project the visualization of chemical components into 2-D or 3-D space for viewing on-screen or through 3-D glasses. The projection may be carried out using metric or non-metric multidimensional scaling or a manifold learning strategy. The visualization can also depict the chemical compounds as network nodes. The node's size, color, or geometric entity (sphere, cube, etc.) may signify a property that must be highlighted. The edges between nodes indicate the interactions/relationships between compounds in the biological space, with the accompanying weight conveying the concept of dissimilatory with respect to all or selected biological attributes.

The network layout in 2-D and 3-D space is determined by executing force-directed graph drawing graph layout algorithms (such as Fruchterman-Reingold, Kamada-Kawai, or others) (Eades, 1984; Fruchterman and Reingold, 1991; Kamada and Kawai, 1989).

Overview of a System in Accordance with Some Embodiments

An example of an interactive visualization system in accordance herewith comprises a high-performance computing unit, a graphical user interface (GUI), and an interactive display module that can convey graphical information in 2D or 3D. The computing unit is equipped with algorithms for processing vectors that describe the biological properties of the analyzed chemical compounds by establishing similarities or dissimilarities between these vectors based on pre-defined distance functions. Furthermore, the computing unit employs algorithms that calculate the position of nodes representing the chemical compounds, so that the relative position of the nodes represents similarity or dissimilarity between the compounds in terms of their biological action. The GUI allows users to input parameters and customize views, while the display module shows the relationships among the compounds.

Integral to the system are data processing and program (algorithmic) resources. A core feature of the system comprises a procedure that computes the similarity between compounds using a pre-defined distance function and a data vector that represents quantified characteristics of the compound in terms of its biological activity. The procedure then positions each compound in space where spatial proximity indicates similarity in biological function.

Interactive 3D Visualization is one of the key features of the system. The display module presents a dynamic, rotatable, and zoomable space where compounds are represented as distinct entities (e.g., spheres, cubes). Each entity's size, color, and texture may correspond to different attributes like molecular weight, solubility, therapeutic class, and multiple other biological or chemical characteristics known from the scientific literature or measured using compound screening techniques such as Cell Health Screen. Connections between entities (edges) indicate known or predicted similarities, with customizable properties such as color and thickness to represent different interaction types or aspects of the similarities or dissimilarities.

User Interaction and Customization are important enabling features. Users can interact with the visualization through the GUI, allowing them to isolate specific compounds, modify views, or focus on particular areas of the multidimensional space. They can also run “what-if” scenarios, adjusting compound properties to predict changes in similarities and, consequently, groupings or clusterings of compounds. The system permits the import and export of data, allowing integration with external databases and research tools.

There many applications. The system is capable of being utilized in pharmaceutical research for comprehending drug interactions, creating innovative therapeutics, and anticipating side effects. In academic research, it assists in clarifying biochemical pathways and compound mechanisms of action. Additionally, this system has applications in educational environments for teaching biochemistry and pharmacology.

While there are many ways to implement systems in according with the herein described inventions and embodiments in preferred implementations, systems feature high-performance computer or multiple computers with substantial processing power, memory, and advanced graphics capabilities. The software is platform-independent and can be installed on various operating systems. It also supports virtual reality (VR) and augmented reality (AR) technologies for immersive experiences. The specific tasks, such as computation of similarities, layout, visualization, and display, can be dedicated to different computers. Therefore, the computational part can be performed on a “server” computer, and visualization and interactions may be performed on a “client” computer. The current implementation employs cloud computing architecture, whereby virtual servers undertake most of the computational tasks, while the client computer is responsible for managing the graphical user interface and presenting the final visualization. The current implementation employs Amazon Cloud computing architecture, whereby virtual servers undertake most of the computational tasks, while the client computer is responsible for managing the graphical user interface and presenting the final visualization. Other architecture or cloud environments are possible.

Illustration of VR implementations of compound universes, filters, database access and the like are depicted in FIGS. 18-26 and described in the brief descriptions thereof above.

### REFERENCES

All references referred to herein, including but not limited to those listed below, are herein incorporated by reference in their entirety, particularly in parts pertinent to the subject matter for which they are cited.
Awale, M., Reymond, J.-L., 2016. Web-based 3D-visualization of the DrugBank chemical space. Journal of Cheminformatics 8, 25. https://doi.org/10.1186/s13321-016-0138-2
Awale, M., van Deursen, R., Reymond, J.-L., 2013. MQN-Mapplet: Visualization of Chemical Space with Interactive Maps of DrugBank. ChEMBL, PubChem, GDB-11, and GDB-13. J. Chem. Inf. Model. 53, 509-518. https://doi.org/10.1021/ci300513m
Bawa, M., Condie. T., Ganesan, P., 2005. LSH forest: self-tuning indexes for similarity search, in: Proceedings of the 14th International Conference on World Wide Web, WWW '05. Association for Computing Machinery. New York. NY. USA, pp. 651-660. https://doi.org/10.1145/1060745.1060840
Bieberich, A. A., Rajwa, B., Irvine. A., Fatig, R. O., Fekete. A., Jin. H., Kutlina, E. Urban, L., 2021. Acute cell stress screen with supervised machine learning predicts cytotoxicity of excipients. Journal of Pharmacological and Toxicological Methods, Seventeenth Annual Themed Issue on Methods in Safety Pharmacology 111, 107088. https://doi.org/10.1016/j.vascn.2021.107088
Borrel, A., Kleinstreuer, N.C., Fourches, D., 2018. Exploring drug space with ChemMaps.com. Bioinformatics 34, 3773-3775. https://doi.org/10.1093/bioinformatics/bty412
Capecchi, A., Reymond. J.-L., 2020. Assigning the origin of microbial natural products by chemical space map and machine learning. Biomolecules 10, 1385. https://doi.org/10.3390/biom101385
Cichocki, A., Amari, S., 2010. Families of alpha- beta- and gamma-divergences: flexible and robust measures of similarities. Entropy 12, 1532-1568. https://doi.org/10.3390/e12061532
Cichocki, A., Cruces, S., Amari. S., 2011. Generalized alpha-beta divergences and their application to robust nonnegative matrix factorization. Entropy 13, 134-170. https://doi.org/10.3390/e13010134
Donmez. A., Rifaioglu, A. S., Acar, A., Dogan, T., Cetin-Atalay, R., Atalay, V., 2020. iBioProVis: interactive visualization and analysis of compound bioactivity space. Bioinformatics 36, 4227-4230. https://doi.org/10.1093/bioinformatics/btaa496
Eades. P., 1984. A heuristic for graph drawing. Congressus numerantium 42, 149-160.
Fruchterman, T. M. J., Reingold, E. M., 1991. Graph drawing by force-directed placement. Software: Practice and Experience 21, 1129-1164. https://doi.org/10.1002/spe.4380211102
Gurcan, F. Cagiltay, N. E., Cagiltay, K., 2021 Mapping human-computer interaction research themes and trends from its existence to today: a topic modeling-based review of past 60 years. International Journal of Human-Computer Interaction 37, 267-280. https://doi.org/10.1080/10447318.2020.1819668
Gütlein, M., Karwath, A., Kramer. S., 2012. CheS-Mapper—Chemical Space Mapping and Visualization in 3D. Journal of Cheminformatics 4, 7. https://doi.org/10.1186/1758-2946-4-7
Jin, X., Awale, M., Zasso, M., Kostro, D., Patiny, L., Reymond, J.-L., 2015. PDB-Explorer: a web-based interactive map of the protein data bank in shape space. BMC Bioinformatics 16, 339. https://doi.org/10.1186/s12859-015-0776-9
Kamada, T., Kawai. S., 1989. An algorithm for drawing general undirected graphs. Information Processing Letters 31, 7-15. https.//doi.org/10.1016/0020-0190(89)90102-6
Karlov, D. S., Sosnin, S., Tetko, I. V., Fedorov. M. V., 2019. Chemical space exploration guided by deep neural networks. RSC Advances 9, 5151-5157. https://doi.org/10.1039/C8RA 10182E
Legetth, O., Rodhe, J., Lang, S., Dhapola, P., Wallergård, M., Soneji, S., 2021. CellexalVR: A virtual reality platform to visualize and analyze single-cell omics data. iScience 24, 103251. https://doi.org/10.1016/j.isci.2021.103251
Liluashvili, V., Kalayci, S., Fluder, E., Wilson. M., Gabow, A., Gümüş, Z. H., 2017. iCAVE: an open source tool for visualizing biomolecular networks in 3D, stereoscopic 3D and immersive 3D. GigaScience 6, gix054. https://doi.org/10.1093/gigascience/gix054
Lu, J., Carlson, H. A., 2016. ChemTreeMvap: an interactive map of biochemical similarity in molecular datasets. Bioinformatics 32, 3584-3592. https://doi.org/10.1093/bioinformatics/btw523
Medina-Franco. J. L., Sanchez-Cruz. N., López-López, E., Díaz-Eufracio, B. I., 2022. Progress on open chemoinformatic tools for expanding and exploring the chemical space. J Comput Aided Mol Des 36, 341-354. https://doi.org/10.1007/s10822-021-00399-1
Probst, D., Reymond, J.-L., 2020. Visualization of very large high-dimensional data sets as minimum spanning trees. Journal of Cheminformatics 12, 12. https.//doi.org/10.1186/s13321-020-0416-x
Probst, D., Reymond, J.-L., 2018. Exploring DrugBank in virtual reality chemical space. J. Chem. Inf. Model. 58, 1731-1735. https://doi.org/10.1021/acs.jcim.8b00402
Rajwa. B., Shankey, V. T., 2021. Identification of functional cell states. US11137388B2.
Reymond, J.-L., 2015. The Chemical Space Project. Acc. Chem. Res. 48, 722-730. https://doi.org/10.1021/ar500432k
Sebastián-Pérez. V., Roca, C., Awale, M., Reymond, J.-L., Martinez. A., Gil, C., Campillo, N. E., 2017. Medicinal and biological chemistry (MBC) library: an efficient source of new hits. J. Chem. Inf. Model. 57, 2143-2151. https://doi.org/10.1021/acs.jcim.7b00401
Stein, D. F., Chen, H., Vinyard, M. E., Qin, Q., Combs. R. D., Zhang. Q., Pinello, L., 2021. singlecellVR: interactive visualization of single-cell data in virtual reality. https://doi.org/10.1101/2020.07.30.229534
Turhan, B., Gümüş, Z. H., 2022. A brave new world: virtual reality and augmented reality in systems biology. Frontiers in Bioinformatics 2. https://doi.org/10.3389/fbinf.2022.873478
Widjojo, E. A., Chinthammit, W., Engelke, U., 2017. Virtual reality-based human-data interaction, in: 2017 International Symposium on Big Data Visual Analytics (BDVA). Presented at the 2017 International Symposium on Big Data Visual Analytics (BDVA), pp. 1-6. https://doi.org/10.1109/BDVA.2017.8114627
Yang, A., Yao. Y., Li, J., Ho, J. W. K., 2018. starmap: Immersive visualisation of single cell data using smartphone-enabled virtual reality. https://doi.org/10.1101/324855

1. A system for displaying properties of compounds, comprising:
(A) One or more selectable databases of information on a plurality of chemical compounds wherein the information in the one or more selectable databases alone or in combination comprises chemical, physical and structural information and biological information for each compound;
(B) A computational device to calculate for compounds selected from one or more of the selectable databases a relative location in a biological parameter space for each of the selected compounds based on biological information for the compound in the database;
(C) A computational device to calculate for compounds selected from one or more of the selectable databases a relative location in a chemical, physical and/or structural properties parameter space for each of the selected compounds based on chemical, physical and/or structural information for the compound in the database;
(D) A computational device to display the parameter spaces of (B) and (C) and to map the selected compounds therein;
(E) One or more computational modules for human-computer interaction effective for users to:
(a) select databases from which information is to be displayed;
(b) select compounds to display from each selected database;
(c) select biological, physical, chemical and/or structural parameter spaces in which compounds will be represented, including spaces based on combinations of the parameters;
(d) select methods for calculating the location and/or distance of compounds in the parameter spaces;
(e) adjust parameters of the parameter space, including scales;
(f) select and display additional information about compounds and
wherein the location of compounds in the biological parameter space is determined by one or more dissimilarity and/or similarity metrics.

2. The system according to claim 1, wherein biological assay measures are stretched between 0 and 1, where 0 denotes lack of response, and 1 denotes maximal observable response, and the similarity between responses is constructed as a distance between these bound response vectors:
$${D}_{1} = {( {\sum }_{i = 1}^{n} {( log ( \frac{1}{1 - {p}_{i}} ) + log ( {p}_{i} ) - log ( \frac{1}{1 - {q}_{i}} ) - log ( {q}_{i} ) )}^{2} )}^{\frac{1}{2}}$$
where p and q are the vectors describing the compounds.

3. (canceled)

4. The system according to claim 1, wherein the similarity between the compounds is computed using AB-divergence formulated as:
$${D}_{AB}^{( α , β )} = \frac{1}{α β} {\sum }_{i = 1}^{n} ( {p}_{i}^{α} {q}_{i}^{β} - \frac{α}{α + β} {p}_{i}^{α + β} - \frac{β}{α + β} {q}_{i}^{α + β} )$$
where α and β are the parameters of the divergence.

5. The system according to claim 1, wherein the similarity between the compounds is computed using AB-divergence formulated as:
$${D}_{AB}^{( α , β )} = {\sum }_{i = 1}^{n} {d}_{AB}^{( α , β )} ( {p}_{i} , {q}_{i} )$$
$$where$$
$${d}_{AB}^{( α , β )} ( {p}_{i} , {q}_{i} ) = \{ \text{} \begin{matrix}- \frac{1}{α β} ( {p}_{i}^{α} {q}_{i}^{β} - \frac{α}{( α + β )} {p}_{i}^{α + β} - \frac{β}{( α + β )} {q}_{i}^{α + β} ) & for α , β , α + β \neq 0 \\ \frac{1}{{α}^{2}} ( {p}_{i}^{α} ln \frac{{p}_{i}^{α}}{{q}_{i}^{α}} - {p}_{i}^{α} + {q}_{i}^{α} ) & for α \neq 0 , β = 0 \\ \frac{1}{{α}^{2}} ( ln \frac{{q}_{i}^{α}}{{p}_{i}^{α}} + {( \frac{{q}_{i}^{α}}{{p}_{i}^{α}} )}^{- 1} - 1 ) & for α = - β \neq 0 \\ \frac{1}{{β}^{2}} ( {q}_{i}^{β} ln \frac{{q}_{i}^{β}}{{p}_{i}^{β}} - {q}_{i}^{β} + {p}_{i}^{β} ) & for α = 0 , β \neq 0 \\ \frac{1}{2} {( ln {p}_{i} - ln {q}_{i} )}^{2} & for α , β = 0\end{matrix} .$$
where p and q are the vectors describing the compounds.

6. A system for enhancing human-computer interaction, comprising:
(A) One or more selectable databases of information on a plurality of chemical compounds wherein the information in the one or more selectable databases alone or in combination comprises chemical, physical and structural information and biological information for each compound;
(B) A computational device to calculate for compounds selected from one or more of the selectable databases a relative location in a biological parameter space for each of the selected compounds based on biological information for the compound in the database;
(C) A computational device to calculate for compounds selected from one or more of the selectable databases a relative location in a chemical, physical and/or structural properties parameter space for each of the selected compounds based on chemical, physical and/or structural information for the compound in the database;
(D) A computational device to display the parameter spaces of (B) and (C) and to map the selected compounds therein;
(E) One or more computational modules for human-computer interaction effective for users to:
(a) select databases from which information is to be displayed;
(b) select compounds to display from each selected database;
(c) select biological, physical, chemical and/or structural parameter spaces in which compounds will be represented, including spaces based on combinations of the parameters;
(d) select methods for calculating the location and/or distance of compounds in the parameter spaces;
(e) adjust parameters of the parameter space, including scales;
(f) select and display additional information about compounds and
wherein the location of compounds in the biological parameter space is determined by one or more dissimilarity and/or a similarity metrics.

7. The system according to claim 6, wherein biological assay measures are stretched between 0 and 1, where 0 denotes lack of response, and 1 denotes maximal observable response, and the similarity between responses is constructed as a distance between these bound response vectors:
$${D}_{1} = {( {\sum }_{i = 1}^{n} {( log ( \frac{1}{1 - {p}_{i}} ) + log ( {p}_{i} ) - log ( \frac{1}{1 - {q}_{i}} ) - log ( {q}_{i} ) )}^{2} )}^{\frac{1}{2}}$$
where p and q are the vectors describing the compounds.

8. (canceled)

9. The system according to claim 6, wherein the similarity between the compounds is computed using AB-divergence formulated as:
$${D}_{AB}^{( α , β )} = \frac{1}{α β} {\sum }_{i = 1}^{n} ( {p}_{i}^{α} {q}_{i}^{β} - \frac{α}{α + β} {p}_{i}^{α + β} - \frac{β}{α + β} {q}_{i}^{α + β} )$$
where α and β are the parameters of the divergence.

10. The system according to claim 6, wherein the similarity between the compounds is computed using AB-divergence formulated alternatively as:
$${D}_{AB}^{( α , β )} = {\sum }_{i = 1}^{n} {d}_{AB}^{( α , β )} ( {p}_{i} , {q}_{i} )$$
$$where$$
$${d}_{AB}^{( α , β )} ( {p}_{i} , {q}_{i} ) = \{ \text{} \begin{matrix}- \frac{1}{α β} ( {p}_{i}^{α} {q}_{i}^{β} - \frac{α}{( α + β )} {p}_{i}^{α + β} - \frac{β}{( α + β )} {q}_{i}^{α + β} ) & for α , β , α + β \neq 0 \\ \frac{1}{{α}^{2}} ( {p}_{i}^{α} ln \frac{{p}_{i}^{α}}{{q}_{i}^{α}} - {p}_{i}^{α} + {q}_{i}^{α} ) & for α \neq 0 , β = 0 \\ \frac{1}{{α}^{2}} ( ln \frac{{q}_{i}^{α}}{{p}_{i}^{α}} + {( \frac{{q}_{i}^{α}}{{p}_{i}^{α}} )}^{- 1} - 1 ) & for α = - β \neq 0 \\ \frac{1}{{β}^{2}} ( {q}_{i}^{β} ln \frac{{q}_{i}^{β}}{{p}_{i}^{β}} - {q}_{i}^{β} + {p}_{i}^{β} ) & for α = 0 , β \neq 0 \\ \frac{1}{2} {( ln {p}_{i} - ln {q}_{i} )}^{2} & for α , β = 0\end{matrix} .$$
and where p and q are the vectors describing the compounds.

11. The system according to claim 1, wherein the visualization and spatial grouping, arrangement or clustering of chemical structures is based on differences and/or similarities in one or more structural or biological features.

12. The system according to claim 1, wherein, one or more physical chemical properties for each chemical compound is combined with one or more biological parameters that are derived from each compound that describe the effect of each compound on intracellular, extracellular or biological proteins, processes or pathways within a cell.

13. The system according to claim 1, wherein one or more chemical substructures is combined with one or more biological parameters that are derived from the effect of a chemical containing those substructures, on intracellular, extracellular or biological proteins, processes or pathways within one or more cells is displayed.

14. The system according to claim 1, wherein the system visualizes and/or displays one or more biological parameters for a compound that are derived from the effect of the chemical containing those substructures on intracellular, extracellular, or biological proteins, processes or pathways within one or more cells.

15. The system according to claim 1, wherein the system visualizes and/or displays one or more acute cellular stress parameters that combine mitochondrial effects with cellular stress effects.

16. The system according to claim 1, wherein the system visualizes and/or displays one or more parameters that are associated with inflammation pathways.

17. The system according to claim 1, wherein the system visualizes and/or displays one or more parameters that are associated with any one or more of DNA Damage Repair, epigenetic regulation, cell cycle and/or GPCR binding and downstream signaling within cells.

18. The system according to claim 1, wherein the system visualizes and/or displays one or more parameters measured in zebrafish.

19. The system according to claim 1, wherein the system visualizes and/or displays one or more parameters that define changes to the morphology of a cell, either direct measurements or calculated measurements that define changes to internal cellular complexity, function, or shape.

20. The system according to claim 1, wherein the system visualizes and/or displays one or more parameters that are collectively termed “cell painting”.

21. The system according to claim 1, wherein the system visualizes and/or displays biological parameters combined with physical-chemical properties and/or chemical structures.

22. The system according to claim 1, wherein the system visually highlights the spatial position of a new, unknown compound within a “universe” of other compounds visualized based on the same chemical and/or biological features, wherein the visualization groups compounds according to a distance function based on calculated similarity and/or difference “distances” between compounds based on the relationship between properties associated with the chemical structure and the effect of that chemical structure on one or more biological processes, pathways or proteins.

23. The system according to claim 1, comprising a virtual reality (VR) display wherein information is displayed and can be manipulated in a virtual reality environment.

24. The system according to claim 23, wherein the VR display comprises one or more of a standalone VR headset, a PC-connected VR headset, a gaming console VR headset, a mixed-reality headset, an enhanced reality headset, a mobile VR headset, a motion controller, a motion platform, a haptic feedback device.

25. The system according to claim 24, wherein the haptic feedback device is any one or more of the following that provide tactile feedback to users: haptic feedback gloves, haptic feedback suits, and haptic feedback vests.

26. The system according to claim 23, wherein compounds are represented as distinct locations in a multiparametric parameter space defined by physical, chemical or biological parameters or combinations thereof, wherein individual compounds can be selected, captured, moved and separated from other compounds, and as desired to outside the parameter space, using physical human motion encoded capturing tools provided by and within the VR subsystem of hardware and software.

27. The system according to claim 23, wherein one or more pluralities of compounds within a parameter space can be selected and then manipulated without affecting other compounds in the parameter space.

28. The system according to claim 27, wherein one or pluralities of compounds can be rotated, enlarged, made smaller, or otherwise visually manipulated using physical human motion encoded capturing tools provided by and within the VR hardware and software.

29. The system according to claim 26, wherein physical, chemical and biological characteristic for a selected compound can be displayed in the multiparametric parameter space using tools provided by and within VR hardware and software.
